(19) United States
(12) Patent Application Publication
(10) Pub. No.: US 2015/0157645 A1
(43) Pub. Date: Jun. 11, 2015

(54) COMBINATION OF A 17-ALPHA-HYDROXYLASE (C17,20-LYASE) INHIBITOR AND A SPECIFIC PI-3K INHIBITOR FOR TREATING A TUMOR DISEASE

(71) Applicants: Samit Hirawat, Chatham, NJ (US); Alessandro Riva, New York, NY (US); Mistry Prakash, Basel (CH)

(72) Inventors: Samit Hirawat, Chatham, NJ (US); Alessandro Riva, New York, NY (US); Mistry Prakash, Basel (CH)

(21) Appl. No.: 14/404,957

(22) PCT Filed: Jun. 4, 2013
(86) PCT No.: PCT/US2013/043989

§ 371 Date: Dec. 2, 2014

Related U.S. Application Data
(60) Provisional application No. 61/815,368, filed on Apr. 24, 2013.

(30) Foreign Application Priority Data
Jun. 6, 2012 (EP) 12305645

(51) Int. Cl. A61K 31/58; A61K 31/444; A61K 31/5377; A61K 31/4745; A61K 31/573

(52) U.S. Cl. CPC A61K 31/58; A61K 31/4745; A61K 31/573; A61K 31/5377; A61K 31/444

(57) ABSTRACT

The present invention relates to a combination which comprises (a) a phosphatidylinositol 3-kinase inhibitor selected from the group consisting of a compound of formula (I) or a compound of formula (II), or pharmaceutically acceptable salt thereof, (b) a 17α-Hydroxylase/C_{17,20}-lyase inhibitor (CYP17 inhibitor), specifically abiraterone acetate and 1-(2-Chloro-pyridin-4-yl)-3-(4-methyl-pyridin-3-yl)-imidazolidin-2-one or pharmaceutically acceptable salt thereof, for simultaneous, separate or sequential use for the treatment of a tumor disease; a pharmaceutical composition comprising such combination; use of such combination for the treatment of a tumor disease; a commercial package or product comprising such combination; and to a method of treating a patient having a tumor disease comprising administration of said combination to a patient in need thereof.

### FIELD OF THE INVENTION

The present invention relates to a combination which comprises (a) a phosphatidylinositol 3-kinase inhibitor selected from the group consisting of a compound of formula (I) or a compound of formula (II), or pharmaceutically acceptable salt thereof, (b) a 17α-Hydroxylase/C_{17,20}-lyase inhibitor (CYP17 inhibitor) or pharmaceutically acceptable salt thereof, for simultaneous, separate or sequential use for the treatment of a tumor disease; a pharmaceutical composition comprising such combination; use of such combination for the treatment of a tumor disease; a commercial package or product comprising such combination; and to a method of treating a patient having a tumor disease comprising administration of said combination to a patient in need thereof.

### BACKGROUND

Epidemiological and experimental studies support an important role for phosphatidylinositol 3-kinases (PI3Ks) in the biology of human cancer. The activation of PI3K, and its downstream effectors, has been clearly validated as an essential step for the initiation and maintenance of the tumorigenic phenotype. Prostate cancers are associated with frequent genetic alterations of the PI3K and/or androgen receptor pathways. Aberrant PI3K signaling is common in prostate cancer with approximately 40% of primary and 70% of metastatic tumors carrying genomic alterations of the PI3K signaling pathway, mostly through loss of PTEN. (Sarker et al, Clinical Cancer Research, Aug. 1, 2009, 15:4799-4805). In preclinical studies, PTEN loss promotes resistance to castration, and PI3K pathway activation in prostate cancer is associated with adverse pathologic and clinical features, and resistance to radiation and chemotherapy. Recent studies disclose that a reciprocal negative feedback regulation of the androgen receptor and PI3K signaling pathways. (Carver et al., Cancer Cell., May 17, 2011, 19(5): 575-586).

Prostate cancer is the most common non-skin cancer in men with over 200,000 new cases diagnosed in 2010 in the U.S., accounting for 28% of new cancer cases in men (Jamal et al, 2010). Prostate cancer is incurable once metastatic and is the second leading cause of cancer death in the U.S. A significant number of patients with prostate cancer develop castrate-resistant disease which has a poor prognosis and poses a therapeutic challenge. Many castrate-resistant tumors remain dependent on androgen receptor signaling.

Recently, abiraterone acetate has been approved for the treatment for castration-resistant prostate cancer. Despite the demonstrated survival benefits with abiraterone acetate in clinical studies, progression-free survival remains relatively short (e.g, 5.6 months) and the proportion of patients with a response (29% Prostate-specific antigen (PSA) or 14% radiologic) is suboptimal, suggesting that primary and/or acquired resistance exists. Treatment options are significantly limited for patients with tumors resistant to therapy with abiraterone acetate. Thus, there is a substantial need for other 17α-Hydroxylase/C_{17,20}-lyase inhibitors (CYP17 inhibitors) and/or more effective therapies based on novel mechanisms of action.

It is now found that the combination comprising a (a) phosphatidylinositol 3-kinase inhibitor selected from the group consisting of a compound of formula (I) or a compound of formula (II), or pharmaceutically acceptable salt thereof, and (b) a 17α-Hydroxylase/C_{17,20}-lyase inhibitor (CYP17 inhibitor) or pharmaceutically acceptable salt thereof, particularly abiraterone acetate or a pharmaceutically acceptable salt thereof, and optionally (c) at least one additional therapeutic agent, particularly a steroid or a pharmaceutically acceptable salt thereof, is effective for the treatment of tumor disease, particularly prostate cancer. It is expected that the anti-proliferative effect of this combination is greater than the maximum effect that can be achieved with either type of ingredient alone.

The compound 1-(2-Chloro-pyridin-4-yl)-3-(4-methyl-pyridin-3-yl)-imidazolidin-2-one or a pharmaceutically acceptable salt thereof is a novel non-steroidal, reversible, potent dual inhibitor of CYP17 and CYP11B2. CYP17 is a key enzyme in the generation of precursors of cortisol and sex steroids. CYP11B2 (aldosterone synthase) is a steroid hydroxylase CYPP450 oxidase enzyme involved in the generation of aldosterone. It is also now found that the combination comprising a (a) phosphatidylinositol 3-kinase inhibitor selected from the group consisting of a compound of formula (I) or a compound of formula (II), or pharmaceutically acceptable salt thereof, and (b) a 17α-Hydroxylase/C_{17,20}-lyase inhibitor (CYP17 inhibitor) or pharmaceutically acceptable salt thereof, particularly 1-(2-Chloro-pyridin-4-yl)-3-(4-methyl-pyridin-3-yl)-imidazolidin-2-one or a pharmaceutically acceptable salt thereof, and optionally (c) at least one additional therapeutic agent, particularly a steroid or a pharmaceutically acceptable salt thereof, is effective for the treatment of tumor disease, particularly prostate cancer. It is expected that the anti-proliferative effect of this combination is greater than the maximum effect that can be achieved with either type of ingredient alone.

### SUMMARY OF THE INVENTION

The present invention pertains to a combination comprising (a) a phosphatidylinositol 3-kinase inhibitor selected from the group consisting of a compound of formula (I) or a compound of formula (II), or pharmaceutically acceptable salt thereof, and (b) a 17α-Hydroxylase/C_{17,20}-lyase inhibitor (CYP17 inhibitor) or pharmaceutically acceptable salt thereof, for simultaneous, separate or sequential use for the treatment of tumor disease, particularly prostate cancer.

In a preferred embodiment, the compound of formula (I) is 2-methyl-2-[4-(3-methyl-2-oxo-8-quinolin-3-yl-2,3-dihydro-imidazo[4,5-c]quinolin-1-yl)-phenyl]-propionitrile (“COMPOUND A”) or its monotosylate salt and 8-(6-methoxy-pyridin-3-yl)-3-methyl-1-(4-piperazin-1-yl-3-trifluoromethyl-phenyl)-1,3-dihydro-imidazo[4,5-c]quinolin-2-one (“COMPOUND B”).

In a preferred embodiment, the compound of formula (II) is 5-(2,6-di-morpholin-4-yl-pyrimidin-4-yl)-4-trifluoromethyl-pyridin-2-ylamine (“COMPOUND C”) or its hydrochloride salt.

In another preferred embodiment, the 17α-Hydroxylase/C_{17,20}-lyase inhibitor is 1-(2-Chloro-pyridin-4-yl)-3-(4-methyl-pyridin-3-yl)-imidazolidin-2-one (“COMPOUND D”) or pharmaceutically acceptable salt thereof.

In a preferred embodiment, the steroid is prednisone or a pharmaceutically acceptable salt thereof.

In one aspect, the present invention provides a pharmaceutical composition comprising a quantity, which is jointly therapeutically effective against a tumor disease, comprising the COMBINATION OF THE INVENTION. In this composition, the combination partners (a) and (b) are administered in a single formulation or unit dosage form by any suitable route. The unit dosage form may also be a fixed combination.

In a further aspect, the present invention provides pharmaceutical compositions separately comprising a quantity, which is jointly therapeutically effective against a tumor disease, of combination partner (a) or combination partner (b) which are administered concurrently but separately, or administered sequentially.

In one aspect, the present invention relates to a method of treating a tumor disease resistant comprising administering to patient in need thereof a COMBINATION OF THE INVENTION in a quantity, which is jointly therapeutically effective against said tumor disease.

In one aspect, the present invention provides a method of inhibiting the formation of metastases in a subject having tumor disease, in particular a prostate cancer, comprising administering to a subject in need thereof an amount of a COMBINATION OF THE INVENTION in a quantity which is therapeutically effective against said tumor disease.

In one aspect, the present invention pertains to the use of a COMBINATION OF THE INVENTION for the treatment of a tumor disease, in particular a prostate cancer, and for the preparation of a medicament for the treatment of a tumor disease.

In one aspect, the present invention pertains to the use of a phosphatidylinositol 3-kinase inhibitor selected from the group consisting of the compound of formula (I), e.g., COMPOUND A or B, or compound of formula (II), e.g., COMPOUND C, or a pharmaceutically acceptable salt thereof in combination with a 17α-Hydroxylase/C_{17,20}-lyase inhibitor or pharmaceutically acceptable salt thereof for the preparation of a medicament for the treatment of a tumor disease, in particular prostate cancer.

In one aspect, the present invention provides a combination comprising (a) a phosphatidylinositol 3-kinase inhibitor selected from the group consisting of the compound of formula (I) or the compound of formula (II) or a pharmaceutically acceptable salt thereof, (b) a 17α-Hydroxylase/C_{17,20}-lyase inhibitor or a pharmaceutically acceptable salt thereof, and (c) at least one additional therapeutic agent. Preferably, the 17α-Hydroxylase/C_{17,20}-lyase inhibitor is a steroid. More preferably, the 17α-Hydroxylase/C_{17,20}-lyase inhibitor is abiraterone acetate or a pharmaceutically acceptable salt thereof.

In one aspect, the present invention provides a combination comprising (a) a phosphatidylinositol 3-kinase inhibitor selected from the group consisting of the compound of formula (I) or the compound of formula (II) or a pharmaceutically acceptable salt thereof, (b) a 17α-Hydroxylase/C_{17,20}-lyase inhibitor, particularly abiraterone acetate, or a pharmaceutically acceptable salt thereof, and (c) at least one additional therapeutic agent, is useful for the treatment of a tumor disease.

In one aspect, the present invention provides a method of treating a patient having a tumor disease which comprises administering to a patient in need thereof a combination comprising (a) a phosphatidylinositol 3-kinase inhibitor selected from the group consisting of the compound of formula (I) or the compound of formula (II) or a pharmaceutically acceptable salt thereof, (b) a 17α-Hydroxylase/C_{17,20}-lyase inhibitor, particularly abiraterone acetate, or a pharmaceutically acceptable salt thereof, and (c) at least one additional therapeutic agent in a quantity which is jointly therapeutically effective against said tumor disease.

In one aspect, the present invention provides a method of inhibiting the formation of metastases in a subject having a tumor disease which comprises administering to a patient in need thereof a combination comprising (a) a phosphatidylinositol 3-kinase inhibitor selected from the group consisting of the compound of formula (I) or the compound of formula (II) or a pharmaceutically acceptable salt thereof, (b) a 17α-Hydroxylase/C_{17,20}-lyase inhibitor, particularly abiraterone acetate, or a pharmaceutically acceptable salt thereof, and (c) at least one additional therapeutic agent in a quantity which is jointly therapeutically effective against said tumor disease.

In one aspect, the present invention provides a combination comprising (a) a phosphatidylinositol 3-kinase inhibitor selected from the group consisting of the compound of formula (I) or the compound of formula (II) or a pharmaceutically acceptable salt thereof, (b) a 17α-Hydroxylase/C_{17,20}-lyase inhibitor or a pharmaceutically acceptable salt thereof, and (c) at least one additional therapeutic agent. Preferably, the 17α-Hydroxylase/C_{17,20}-lyase inhibitor is a steroid. More preferably, the 17α-Hydroxylase/C_{17,20}-lyase inhibitor is 1-(2-Chloro-pyridin-4-yl)-3-(4-methyl-pyridin-3-yl)-imidazolidin-2-one or a pharmaceutically acceptable salt thereof.

In one aspect, the present invention provides a combination comprising (a) a phosphatidylinositol 3-kinase inhibitor selected from the group consisting of the compound of formula (I) or the compound of formula (II) or a pharmaceutically acceptable salt thereof, (b) a 17α-Hydroxylase/C_{17,20}-lyase inhibitor, particularly 1-(2-Chloro-pyridin-4-yl)-3-(4-methyl-pyridin-3-yl)-imidazolidin-2-one, or a pharmaceutically acceptable salt thereof, and (c) at least one additional therapeutic agent, is useful for the treatment of a tumor disease.

In one aspect, the present invention provides a method of treating a patient having a tumor disease which comprises administering to a patient in need thereof a combination comprising (a) a phosphatidylinositol 3-kinase inhibitor selected from the group consisting of the compound of formula (I) or the compound of formula (II) or a pharmaceutically acceptable salt thereof, (b) a 17α-Hydroxylase/C_{17,20}-lyase inhibitor, particularly 1-(2-Chloro-pyridin-4-yl)-3-(4-methyl-pyridin-3-yl)-imidazolidin-2-one, or a pharmaceutically acceptable salt thereof, and (c) at least one additional therapeutic agent in a quantity which is jointly therapeutically effective against said tumor disease.

In one aspect, the present invention provides a method of inhibiting the formation of metastases in a subject having a tumor disease which comprises administering to a patient in need thereof a combination comprising (a) a phosphatidylinositol 3-kinase inhibitor selected from the group consisting of the compound of formula (I) or the compound of formula (II) or a pharmaceutically acceptable salt thereof, (b) a 17α-Hydroxylase/C_{17,20}-lyase inhibitor, particularly 1-(2-Chloro-pyridin-4-yl)-3-(4-methyl-pyridin-3-yl)-imidazolidin-2-one, or a pharmaceutically acceptable salt thereof, and (c) at least one additional therapeutic agent in a quantity which is jointly therapeutically effective against said tumor disease.

In one aspect, the present invention provides a commercial package comprising as active ingredients COMBINATION OF THE INVENTION, together with instructions for the simultaneous, separate or sequential use thereof in the treatment of a tumor disease, particularly prostate cancer.

### BRIEF DESCRIPTION OF THE DRAWINGS

FIG. 1a summarizes the tumor volume as a function of days post treatment using a phosphatidylinositol 3-kinase inhibitor (30 mg)/CYP17 inhibitor (300 mg) combination in a human prostate VCap xenograft model in SCID mice.

FIG. 1 b summarizes corresponding weight loss in the SCID mice as a function of days post treatment using a phosphatidylinositol 3-kinase inhibitor (30 mg)/CYP17 (300 mg) inhibitor combination.

FIG. 2a summarizes the tumor volume as a function of days post treatment using a phosphatidylinositol 3-kinase inhibitor (20 mg)/CYP17 inhibitor (300 mg) combination in a human prostate VCap xenograft model in SCID mice.

FIG. 2b summarizes corresponding weight loss in the SCID mice as a function of days post treatment using a phosphatidylinositol 3-kinase inhibitor (20 mg)/CYP17 (300 mg) inhibitor combination.

FIG. 3a summarizes the tumor volume as a function of days post treatment using a phosphatidylinositol 3-kinase inhibitor (30 mg)/CYP17 inhibitor (300 mg) combination in a human prostate LNCap xenograft model in SCID mice.

FIG. 3b summarizes corresponding weight loss in the SCID mice as a function of days post treatment using a phosphatidylinositol 3-kinase inhibitor (30 mg)/CYP17 (300 mg) inhibitor combination.

### DETAILED DESCRIPTION

The present invention pertains to a pharmaceutical combination comprising (a) a phosphatidylinositol 3-kinase inhibitor selected from the group consisting of a compound of formula (I) or a compound of formula (II), or pharmaceutically acceptable salt thereof, and (b) a 17α-Hydroxylase/C_{17,20}-lyase inhibitor (CYP17 inhibitor) or pharmaceutically acceptable salt thereof, for simultaneous, separate or sequential use for the treatment of tumor disease, particularly prostate cancer.

The general terms used herein are defined with the following meanings, unless explicitly stated otherwise:

The terms “comprising” and “including” are used herein in their open-ended and non-limiting sense unless otherwise noted.

The terms “a” and “an” and “the” and similar references in the context of describing the invention (especially in the context of the following claims) are to be construed to cover both the singular and the plural, unless otherwise indicated herein or clearly contradicted by context. Where the plural form is used for compounds, salts, and the like, this is taken to mean also a single compound, salt, or the like.

The term “combination” or “pharmaceutical combination” is defined herein to refer to either a fixed combination in one dosage unit form, a non-fixed combination or a kit of parts for the combined administration where the phosphatidylinositol 3-kinase inhibitor and the 17α-Hydroxylase/C_{17,20}-lyase inhibitor (CYP17 inhibitor) or pharmaceutically acceptable salt thereof may be administered independently at the same time or separately within time intervals that allow that the combination partners show a cooperative, e.g., synergistic, effect.

The term “fixed combination” means that the active ingredients, e.g. the phosphatidylinositol 3-kinase inhibitor and the 17α-Hydroxylase/C_{17,20}-lyase inhibitor (CYP17 inhibitor) are administered to a patient simultaneously in the form of a single entity or dosage form.

The term “non-fixed combination” means that the active ingredients, e.g. the phosphatidylinositol 3-kinase inhibitor and the 17α-Hydroxylase/C_{17,20}-lyase inhibitor (CYP17 inhibitor) or pharmaceutically acceptable salt thereof, are both administered to a patient as separate entities or dosage forms either simultaneously, concurrently or sequentially with no specific time limits, wherein such administration provides therapeutically effective levels of the three compounds in the body of the subject, e.g., a mammal or human, in need thereof.

The term “a phosphatidylinositol 3-kinase inhibitor” or “PI3K inhibitor” is defined herein to refer to a compound which targets, decreases or inhibits phosphatidylinositol 3-kinase. Phosphatidylinositol 3-kinase activity has been shown to increase in response to a number of hormonal and growth factor stimuli, including insulin, platelet-derived growth factor, insulin-like growth factor, epidermal growth factor, colony-stimulating factor, and hepatocyte growth factor, and has been implicated in processes related to cellular growth and transformation.

The term “pharmaceutical composition” is defined herein to refer to a mixture or solution containing at least one therapeutic agent to be administered to a subject, e.g., a mammal or human, in order to treat a particular disease or condition affecting the subject thereof.

The term “pharmaceutically acceptable” is defined herein to refer to those compounds, materials, compositions and/or dosage forms, which are, within the scope of sound medical judgment, suitable for contact with the tissues a subject, e.g., a mammal or human, without excessive toxicity, irritation allergic response and other problem complications commensurate with a reasonable benefit/risk ratio.

The terms “co-administration” and “combined administration” as used herein are defined to encompass the administration of the selected therapeutic agents to a single subject, e.g., a mammal or human, and are intended to include treatment regimens in which the agents are not necessarily administered by the same route of administration or at the same time.

The term “treating” or “treatment” as used herein comprises a treatment relieving, reducing or alleviating at least one symptom in a subject or effecting a delay of progression of a disease. For example, treatment can be the diminishment of one or several symptoms of a disorder or complete eradication of a disorder, such as cancer. Within the meaning of the present invention, the term “treat” also denotes to arrest, delay the onset (i.e., the period prior to clinical manifestation of a disease) and/or reduce the risk of developing or worsening a disease.

The term “jointly therapeutically active” or “joint therapeutic effect” as used herein means that the therapeutic agents may be given separately (in a chronologically staggered manner, especially a sequence-specific manner) in such time intervals that they prefer, in the warm-blooded animal, especially human, to be treated, still show a (preferably synergistic) interaction (joint therapeutic effect). Whether this is the case can, inter alia, be determined by following the blood levels, showing that both therapeutic agents are present in the blood of the human to be treated at least during certain time intervals.

The term “pharmaceutically effective amount” or “clinically effective amount” of a combination of therapeutic agents is an amount sufficient to provide an observable improvement over the baseline clinically observable signs and symptoms of the tumor disease treated with the combination.

The term “synergistic effect” as used herein refers to action of two agents such as, for example, (a) a compound of formula (I), e.g., COMPOUND A or a pharmaceutically acceptable salt thereof, and a 17α-Hydroxylase/C_{17,20}-lyase inhibitor or pharmaceutically acceptable salt thereof, e.g., abiraterone acetate or a pharmaceutically acceptable salt thereof, or (b) or for example, a compound of formula (II), e.g, COMPOUND C or a pharmaceutically acceptable salt thereof and a 17α-Hydroxylase/C_{17,20}-lyase inhibitor or pharmaceutically acceptable salt thereof, e.g, abiraterone acetate or a pharmaceutically acceptable salt thereof or 1-(2-Chloro-pyridin-4-yl)-3-(4-methyl-pyridin-3-yl)-imidazolidin-2-one or a pharmaceutically acceptable salt thereof, producing an effect, for example, slowing the symptomatic progression of a proliferative disease, particularly cancer, or symptoms thereof, which is greater than the simple addition of the effects of each drug administered by themselves. A synergistic effect can be calculated, for example, using suitable methods such as the Sigmoid-Emax equation (Holford, N. H. G. and Scheiner, L. B., Clin. Pharmacokinet. 6: 429-453 (1981)), the equation of Loewe additivity (Loewe, S. and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied to experimental data to generate a corresponding graph to aid in assessing the effects of the drug combination. The corresponding graphs associated with the equations referred to above are the concentration-effect curve, isobologram curve and combination index curve, respectively.

The term “subject” or “patient” as used herein includes animals, which are capable of suffering from or afflicted with a tumor disease or any disorder involving, directly or indirectly, a tumor. Examples of subjects include mammals, e.g., humans, dogs, cows, horses, pigs, sheep, goats, cats, mice, rabbits, rats and transgenic non-human animals. In the preferred embodiment, the subject is a human, e.g., a human suffering from, at risk of suffering from, or potentially capable of suffering from a tumor disease.

The term about” or “approximately” shall have the meaning of within 10%, more preferably within 5%, of a given value or range.

Combinations of the present invention include a phosphatidylinositol 3-kinase inhibitor (PI3K) compound selected from the group consisting of compounds of formula (I) or compounds of formula (II) or pharmaceutically acceptable salts thereof.

WO2006/122806 and WO2008/103636 describe imidazoquinoline derivatives, which have been found to inhibit the activity of phosphatidylinositol-3-kinase (PI3K) and mammalian target of rapamycin (mTOR). Specific imidazoquinoline derivatives which are suitable for the present invention, their preparation and suitable pharmaceutical formulations containing the same are described in WO2006/122806 and WO2008/103636 and include compounds of formula (I)

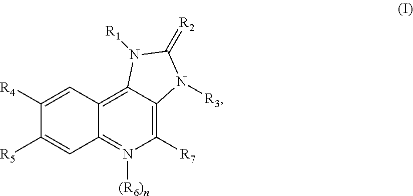

wherein
R₁ is naphthyl or phenyl wherein said phenyl is substituted by one or two substituents independently selected from the group consisting of Halogen; lower alkyl unsubstituted or substituted by halogen, cyano, imidazolyl or triazolyl; cycloalkyl; amino substituted by one or two substituents independently selected from the group consisting of lower alkyl, lower alkyl sulfonyl, lower alkoxy and lower alkoxy lower alkylamino; piperazinyl unsubstituted or substituted by one or two substituents independently selected from the group consisting of lower alkyl and lower alkyl sulfonyl; 2-oxo-pyrrolidinyl; lower alkoxy lower alkyl; imidazolyl; pyrazolyl; and triazolyl;
R₂ is O or S;
R₃ is lower alkyl;
R₄ is pyridyl unsubstituted or substituted by halogen, cyano, lower alkyl, lower alkoxy or piperazinyl unsubstituted or substituted by lower alkyl; pyrimidinyl unsubstituted or substituted by lower alkoxy; quinolinyl unsubstituted or substituted by halogen; quinoxalinyl; or phenyl substituted with alkoxy
R₅ is hydrogen or halogen;
n is 0 or 1;
R₆ is oxido;
with the proviso that if n=1, the N-atom bearing the radical R₆ has a positive charge;
R₇ is hydrogen or amino.

The radicals and symbols as used in the definition of a compound of formula (I) have the meanings as disclosed in WO2006/122806 which publication is hereby incorporated into the present application by reference in its entirety.

A phosphatidylinositol 3-kinase inhibitor compound of formula (I) may be present in the combination in the form of the free base or a pharmaceutically acceptable salt thereof. Suitable salts of the compounds of formula (I) include those formed, for example, as acid addition salts, preferably with organic or inorganic acids. Suitable inorganic acids are, for example, halogen acids, such as hydrochloric acid, sulfuric acid, or phosphoric acid. Suitable organic acids are, for example, carboxylic, phosphonic, sulfonic or sulfamic acids, for example acetic acid, pro [symbol] pionic acid, octanoic acid, decanoic acid, dodecanoic acid, glycolic acid, lactic acid, fumaric acid, succinic acid, malonic acid, adipic acid, pimelic acid, suberic acid, azelaic acid, malic acid, tartaric acid, citric acid, amino acids, such as glutamic acid or aspartic acid, maleic acid, hydroxymaleic acid, methylmaleic acid, cyclohexanecarboxylic acid, adamantanecarboxylic acid, benzoic acid, salicylic acid, 4-aminosalicylic acid, phthalic acid, phenylacetic acid, mandelic acid, cinnamic acid, methane- or ethane-sulfonic acid, 2-hydroxyethanesulfonic acid, etha [symbol] ne-1,2-disulfonic acid, benzenesulfonic acid, 4-toluenesulfonic acid, 2-naphthalenesulfonic acid, 1,5-naphthalene-disulfonic acid, 2- or 3-methylbenzenesulfonic acid, methylsulfuric acid, ethylsulfuric acid, dodecylsulfuric acid, N-cyclohexylsulfamic acid, N-methyl-, N-ethyl- or N-propyl-sulfamic acid, or other organic protonic acids, such as ascorbic acid.

Preferred compounds of formula (I) for use in the combination of the present invention are 2-methyl-2-[4-(3-methyl-2-oxo-8-quinolin-3-yl-2,3-dihydro-imidazo[4,5-c]quinolin-1-yl)-phenyl]propionitrile (“COMPOUND A”) or its monotosylate salt and 8-(6-methoxy-pyridin-3-yl)-3-methyl-1-(4-piperazin-1-yl-3-trifluoromethyl-phenyl)-1,3-dihydro-imidazo[4,5-c]quinolin-2-one (“COMPOUND B”). The synthesis of 2-methyl-2-[4-(3-methyl-2-oxo-8-quinolin-3-yl-2,3-dihydro-imidazo[4,5-c]quinolin-1-yl)-phenyl]-propionitrile and its monotosylate is for instance described in WO2006/122806 as Examples 7 and 152-3 respectively. The synthesis of 8-(6-methoxy-pyridin-3-yl)-3-methyl-1-(4-piperazin-1-yl-3-trifluoromethyl-phenyl)-1,3-dihydro-imidazo[4,5-c]quinolin-2-one is for instance described in WO2006/122806 as Example 86. In one preferred embodiment, the compound of formula (I) is 2-methyl-2-[4-(3-methyl-2-oxo-8-quinolin-3-yl-2,3-dihydro-imidazo[4,5-c]quinolin-1-yl)-phenyl]propionitrile (“COMPOUND A”) or its monotosylate salt.

WO07/084786 describes pyrimidine derivatives, which have been found to inhibit the activity of phosphatidylinositol 3-kinase (PI3K). Specific phosphatidylinositol 3-kinase (PI3K) inhibitors suitable for the present invention, their preparation and suitable pharmaceutical formulations containing the same are described in WO07/084786 and include compounds of formula (II):

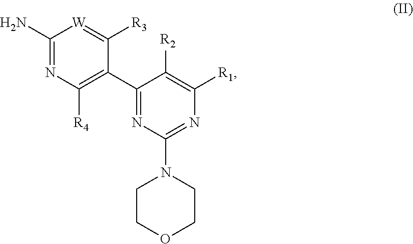

wherein W is CR_{w} or N, wherein

R_{w} is selected from the group consisting of:
(1) hydrogen,
(2) cyano,
(3) halogen,
(4) methyl,
5) trifluoromethyl,
(6) sulfonamide;

R₁ is selected from the group consisting of:
(1) hydrogen,
(2) cyano,
(3) nitro,
(4) halogen,
(5) substituted and unsubstituted alkyl,
(6) substituted and unsubstituted alkenyl,
(7) substituted and unsubstituted alkynyl,
(8) substituted and unsubstituted aryl,
(9) substituted and unsubstituted heteroaryl,
(10) substituted and unsubstituted heterocyclyl,
(11) substituted and unsubstituted cycloalkyl,
(12) —COR₁ₐ,
(13) —CO₂R₁ₐ,
(14) —CONR₁ₐR_{1b},
(15) —NR₁ₐR_{1b},
(16) —NR₁ₐCOR_{1b},
(17) —NR₁ₐSO₂R_{1b},
(18) —OCOR₁ₐ,
(19) —OR₁ₐ,
(20) —SR₁ₐ,
(21) —SOR₁ₐ,
(23) —SO₂NR_{Ia}R_{1b} wherein

R₁ₐ, and R_{1b} are independently selected from the group consisting of:
(a) hydrogen,
(b) substituted or unsubstituted alkyl,
(c) substituted and unsubstituted aryl,
(d) substituted and unsubstituted heteroaryl,
(e) substituted and unsubstituted heterocyclyl, and
(f) substituted and unsubstituted cycloalkyl;

R₂ is selected from the group consisting of:
(1) hydrogen,
(2) cyano,
(3) nitro,
(4) halogen,
(5) hydroxy,
(6) amino,
(7) substituted and unsubstituted alkyl,
(8) —COR₂ₐ, and
(9) —NR₂ₐCOR_{2b}, wherein

R₂ₐ, and R_{2b} are independently selected from the group consisting of:
(a) hydrogen, and
(b) substituted or unsubstituted alkyl;

R₃ is selected from the group consisting of:
(1) hydrogen,
(2) cyano,
(3) nitro,
(4) halogen,
(5) substituted and unsubstituted alkyl,
(6) substituted and unsubstituted alkenyl,
(7) substituted and unsubstituted alkynyl,
(8) substituted and unsubstituted aryl,
(9) substituted and unsubstituted heteroaryl,
(10) substituted and unsubstituted heterocyclyl,
(11) substituted and unsubstituted cycloalkyl,
(12) —COR₃ₐ,
(14) —NR₃ₐR_{3b}
(13) —NR₃ₐCOR_{3b},
(15) —NR₃ₐSO₂R_{3b},
(16) —OR₃ₐ,
(17) —SR₃ₐ,
(18) —SOR₃ₐ,
(19) —SO₂R₃ₐ, wherein

R₃ₐ, and R_{3b} are independently selected from the group consisting of:
(a) hydrogen,
(b) substituted or unsubstituted alkyl,
(c) substituted and unsubstituted aryl,
(d) substituted and unsubstituted heteroaryl,
(e) substituted and unsubstituted heterocyclyl, and
(f) substituted and unsubstituted cycloalkyl; and

R₄ is selected from the group consisting of
(1) hydrogen, and
(2) halogen.

The radicals and symbols as used in the definition of a compound of formula (II) have meanings as disclosed in WO07/084786 which publication is hereby incorporated into the present application by reference in its entirety.

The phosphatidylinositol 3-kinase inhibitor compound of formula (II) may be present in the combination in the form of the free base or a pharmaceutically acceptable salt thereof. Such salts can be prepared in situ during the final isolation and purification of the compounds, or by separately reacting the base or acid functions with a suitable organic or inorganic acid or base, respectively. Suitable salts of the compound of formula (II) include but are not limited to the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemi-sulfate, heptanoate, hexanoate, fumarate, hydrochloride, hydrobromide, hydroiodide, 2 hydroxyethanesulfonate, lactate, maleate, methanesulfonate, nicotinate, 2 naphth-alenesulfonate, oxalate, pamoate, pectinate, persulfate, 3 phenylproionate, picrate, pivalate, propionate, succinate, sulfate, tartrate, thiocyanate, p toluenesulfonate, and undecanoate. Also, the basic nitrogen-containing groups can be quaternized with such agents as alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl, and stearyl chlorides, bromides and iodides, aralkyl halides like benzyl and phenethyl bromides, and others.

Examples of acids that may be employed to form pharmaceutically acceptable acid addition salts include such inorganic acids as hydrochloric acid, hydroboric acid, nitric acid, sulfuric acid and phosphoric acid and such organic acids as formic acid, acetic acid, trifluoroacetic acid, fumaric acid, tartaric acid, oxalic acid, maleic acid, methanesulfonic acid, succinic acid, malic acid, methanesulfonic acid, benzenesulfonic acid, and p toluenesulfonic acid, citric acid, and acidic amino acids such as aspartic acid and glutamic acid.

Pharmaceutically acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, piperazine, pyridine, picoline, triethanolamine and the like, and basic amino acids such as arginine, lysine and ornithine.

A preferred compound of formula (II) for use in the combination of the present invention is the phosphatidylinositol 3-kinase (PI3K) inhibitor 5-(2,6-di-morpholin-4-yl-pyrimidin-4-yl)-4-trifluoromethyl-pyridin-2-ylamine (hereinafter “COMPOUND C”) or its hydrochloride salt. The synthesis of COMPOUND C is described in WO 2007/084786 as Example 10, the contents of which are incorporated herein by reference.

Combinations of the present invention further include a 17α-Hydroxylase/C_{17,20}-lyase inhibitor or pharmaceutically acceptable salt thereof. Examples of suitable 17α-Hydroxylase/C_{17,20}-lyase inhibitors include, but are not limited to, 1-(2-Chloro-pyridin-4-yl)-3-(4-methyl-pyridin-3-yl)-imidazolidin-2-one, ketoconazole, abiraterone acetate, abiraterone, and TOK-001 (Galeterone) or a pharmaceutically acceptable salt thereof.

Preferably, the 17α-Hydroxylase/C_{17,20}-lyase inhibitor or pharmaceutically acceptable salt thereof is abiraterone acetate or a pharmaceutically acceptable salt thereof. Abiraterone acetate (CAS Registry NO. 154229-18-2), is a compound known by the chemical name (3S, 10R, 13S)-10,13-dimethyl-17-pyridin-3-yl-2,3,4,7,8,9,11,12,14,15-decahydro-1H-cyclopenta[a]phenanthren-3-yl]acetate) and the formula (III),

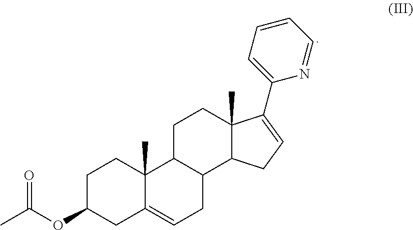 [SMILES: CC(=O)O[C@H]1CC[C@@]2(C)C(=CCC3C2CC[C@]2(C)C(c4ccccn4)=CCC32)C1]

Abiraterone acetate is commercially available as ZYTIGA® from Janssen Biotech, Inc. and disclosed in PCT International Application WO 93/20097, the contents of which are incorporated herein by reference. Abiraterone acetate is converted in vivo to abiraterone, an androgen biosynthesis inhibitor, that inhibits CYP17 (17α-hydroxylase/C_{17,20}-lyase).

Preferred salts of abiraterone acetate include but are not limited to acetates, citrates, lactates, alkanesulfonates (including methane-sulfonates or mesylates) and tartrates. It is understood by one of skill in the art that such salts can be prepared in situ during the final isolation and purification of the compounds, or by separately reacting the base or acid functions with a suitable organic or inorganic acid or base, respectively.

Preferably, the 17α-Hydroxylase/C_{17,20}-lyase inhibitor or pharmaceutically acceptable salt thereof is 1-(2-Chloro-pyridin-4-yl)-3-(4-methyl-pyridin-3-yl)-imidazolidin-2-one or a pharmaceutically acceptable salt thereof. The compound 1-(2-Chloro-pyridin-4-yl)-3-(4-methyl-pyridin-3-yl)-imidazolidin-2-one or a pharmaceutically acceptable salt thereof is a novel non-steroidal, reversible, potent dual inhibitor of CYP17 and CYP11B2, of the formula:

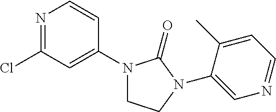 [SMILES: Cc1ccncc1N1CCN(c2ccnc(Cl)c2)C1=O]

and is disclosed in U.S. Pat. No. 8,263,635. CYP17 is a key enzyme in the generation of precursors of cortisol and sex steroids. CYP11B2 (aldosterone synthase) is a steroid hydroxylase CYPP450 oxidase enzyme involved in the generation of aldosterone. The compound is usefully employed in the form of the free base or any of the conventional pharmaceutically acceptable salts thereof, including but not limited to, acetates, citrates, lactates, alkanesulfonates (including methane-sulfonates or mesylates) and tartrates. It is understood by one of skill in the art that such salts can be prepared in situ during the final isolation and purification of the compounds, or by separately reacting the base functions with a suitable organic or inorganic acid, respectively.

A combination comprising (a) a phosphatidylinositol 3-kinase inhibitor selected from the group consisting of a compound of formula (I) or a compound of formula (II), or pharmaceutically acceptable salt thereof, and (b) a 17α-Hydroxylase/C_{17,20}-lyase inhibitor or pharmaceutically acceptable salt thereof, will be referred to hereinafter as a COMBINATION OF THE INVENTION.

In one preferred embodiment of the present invention, the combination comprises (a) a phosphatidylinositol 3-kinase inhibitor selected from the group consisting of COMPOUND A, COMPOUND B or COMPOUND C or a pharmaceutically acceptable salt thereof, (b) abiraterone acetate or a pharmaceutically acceptable salt thereof.

In another preferred embodiment of the present invention the combination comprises (a) a phosphatidylinositol 3-kinase inhibitor COMPOUND A or a pharmaceutically acceptable salt thereof, and (b) abiraterone acetate or a pharmaceutically acceptable salt thereof.

In another preferred embodiment of the present invention, the combination comprises (a) a phosphatidylinositol 3-kinase inhibitor COMPOUND C or a pharmaceutically acceptable salt thereof, (b) abiraterone acetate or a pharmaceutically acceptable salt thereof.

In one preferred embodiment of the present invention, the combination comprises (a) a phosphatidylinositol 3-kinase inhibitor selected from the group consisting of COMPOUND A, COMPOUND B or COMPOUND C or a pharmaceutically acceptable salt thereof, (b) 1-(2-Chloro-pyridin-4-yl)-3-(4-methyl-pyridin-3-yl)-imidazolidin-2-one (COMPOUND D) or a pharmaceutically acceptable salt thereof.

In another preferred embodiment of the present invention the combination comprises (a) a phosphatidylinositol 3-kinase inhibitor COMPOUND A or a pharmaceutically acceptable salt thereof, and (b) 1-(2-Chloro-pyridin-4-yl)-3-(4-methyl-pyridin-3-yl)-imidazolidin-2-one or a pharmaceutically acceptable salt thereof.

In another preferred embodiment of the present invention, the combination comprises (a) a phosphatidylinositol 3-kinase inhibitor COMPOUND C or a pharmaceutically acceptable salt thereof, (b) 1-(2-Chloro-pyridin-4-yl)-3-(4-methyl-pyridin-3-yl)-imidazolidin-2-one or a pharmaceutically acceptable salt thereof.

The nature of tumor diseases like tumor diseases is multifactorial. Under certain circumstances, drugs with different mechanisms of action may be combined. However, just considering any combination of drugs having different mode of action does not necessarily lead to combinations with advantageous effects.

It has been found that the administration of the COMBINATION OF THE INVENTION may be used to treat a subject having a tumor disease, particularly prostate cancer. In the present invention, the administration of the COMBINATION OF THE INVENTION results in a more beneficial treatment, e.g, synergistic or improved anti-proliferative effect, e.g., with regard to the delay of progression of tumor disease or with regard to a change in tumor volume, as compared to either monotherapy.

In a preferred embodiment, the COMBINATIONS OF THE INVENTION is particularly suitable for the treatment of patients with prostate cancer (including but not limited to castration resistant prostate cancer).

In accordance with the present invention, a patient having a tumor disease, particularly prostate cancer, may be separately, simultaneously or sequentially administered (a) a phosphatidylinositol 3-kinase inhibitor selected from the group consisting of a compound of formula (I) or a compound of formula (II), or pharmaceutically acceptable salt thereof, and (b) a 17α-Hydroxylase/C_{17,20}-lyase inhibitor or pharmaceutically acceptable salt thereof for the treatment of said tumor.

Examples of tumor diseases suitable for treatment with the COMBINATION OF THE INVENTION include, but not limited to, benign or malignant tumors, carcinoma of the brain, kidney, liver, bladder, breast, gastric, ovaries, colon, rectum, prostate, pancreas, lung, vagina or thyroid, or gastrointestinal cancers.

In a preferred embodiment, the tumor treated is prostate cancer.

In another preferred embodiment, the tumor treated is prostate cancer resistant to treatment with a 17α-Hydroxylase/C_{17,20}-lyase inhibitor, particularly abiraterone acetate, or a pharmaceutically acceptable salt thereof. In another preferred embodiment, the tumor treated is castration-resistant prostate cancer which is resistant to treatment with abiraterone acetate or a pharmaceutically acceptable salt thereof.

In another preferred embodiment, the tumor treated is prostate cancer resistant to treatment with a 17α-Hydroxylase/C_{17,20}-lyase inhibitor, particularly 1-(2-Chloro-pyridin-4-yl)-3-(4-methyl-pyridin-3-yl)-imidazolidin-2-one, or a pharmaceutically acceptable salt thereof. In another preferred embodiment, the tumor treated is castration-resistant prostate cancer which is resistant to treatment with 1-(2-Chloro-pyridin-4-yl)-3-(4-methyl-pyridin-3-yl)-imidazolidin-2-one or a pharmaceutically acceptable salt thereof.

The administration of a COMBINATION OF THE INVENTION may result not only in a beneficial effect, e.g. therapeutic effect as compared to monotherapy of compound of formula (I), compound of formula (II) and/or a 17α-Hydroxylase/C_{17,20}-lyase inhibitor or pharmaceutically acceptable salt thereof, e.g, a synergistic therapeutic effect, e.g. with regard to alleviating, delaying progression of or inhibiting the symptoms, but also in further surprising beneficial effects, e.g. fewer side-effects, an improved quality of life or a decreased morbidity, compared with a monotherapy applying only one of the pharmaceutically active ingredients used in the combination of the invention.

A further benefit is that lower doses of the active ingredients of the COMBINATION OF THE INVENTION can be used, for example, that the dosages need not only often be smaller, but are also applied less frequently, or can be used in order to diminish the incidence of side-effects observed with one of the combination partners alone. This is in accordance with the desires and requirements of the patients to be treated.

It can be shown by established test models that a COMBINATION OF THE INVENTION results in the beneficial effects described herein before. The person skilled in the art is fully enabled to select a relevant test model to prove such beneficial effects. The pharmacological activity of a COMBINATION OF THE INVENTION may, for example, be demonstrated in a clinical study or in a test procedure as essentially described hereinafter.

Suitable clinical studies are in particular, for example, open label, dose escalation studies in patients with a tumor disease, particularly prostate cancer (including but not limited to castration resistant prostate cancer), resistant to the treatment of abiraterone acetate or a pharmaceutically acceptable salt thereof. Such studies prove in particular the synergism of the therapeutic agents of the combination of the invention. The beneficial effects on tumor diseases may be determined directly through the results of these studies which are known as such to a person skilled in the art. Such studies may be, in particular, be suitable to compare the effects of a monotherapy using either therapeutic agent and a combination of the invention. In one embodiment, the dose of the phosphatidylinositol 3-kinase inhibitor selected from the group consisting of the compound of formula (I), e.g., COMPOUND A or B, or compound of formula (II), e.g., COMPOUND C, is escalated until the Maximum Tolerated Dosage is reached, and the 17α-Hydroxylase/C_{17,20}-lyase inhibitor, e.g, abiraterone acetate, is administered with a fixed dose. Alternatively, phosphatidylinositol 3-kinase inhibitor selected from the group consisting of the compound of formula (I), e.g., COMPOUND A or B, or compound of formula (II), e.g., COMPOUND C, may be administered in a fixed dose and the dose of the 17α-Hydroxylase/C_{17,20}-lyase inhibitor, e.g., abiraterone acetate may be escalated. Each patient may receive doses of the phosphatidylinositol 3-kinase inhibitor either daily or intermittently. The efficacy of the treatment may be determined in such studies, e.g., after 12, 18 or 24 weeks by evaluation of symptom scores every 6 weeks.

In another embodiment, the dose of the phosphatidylinositol 3-kinase inhibitor selected from the group consisting of the compound of formula (I), e.g., COMPOUND A or B, or compound of formula (II), e.g., COMPOUND C, is escalated until the Maximum Tolerated Dosage is reached, and the 17α-Hydroxylase/C_{17,20}-lyase inhibitor, e.g, 1-(2-Chloro-pyridin-4-yl)-3-(4-methyl-pyridin-3-yl)-imidazolidin-2-one, is administered with a fixed dose. Alternatively, phosphatidylinositol 3-kinase inhibitor selected from the group consisting of the compound of formula (I), e.g., COMPOUND A or B, or compound of formula (II), e.g., COMPOUND C, may be administered in a fixed dose and the dose of the 17α-Hydroxylase/C_{17,20}-lyase inhibitor, e.g., 1-(2-Chloro-pyridin-4-yl)-3-(4-methyl-pyridin-3-yl)-imidazolidin-2-one may be escalated. Each patient may receive doses of the phosphatidylinositol 3-kinase inhibitor either daily or intermittently. The efficacy of the treatment may be determined in such studies, e.g., after 12, 18 or 24 weeks by evaluation of symptom scores every 6 weeks.

In one aspect, the invention provides a pharmaceutical composition comprising a quantity, which is jointly therapeutically effective against a tumor disease, comprising the COMBINATION OF THE INVENTION. In this composition, the combination partners (a) and (b) are administered in a single formulation or unit dosage form by any suitable route. The unit dosage form may also be a fixed combination.

In a further aspect, the invention provides pharmaceutical compositions separately comprising a quantity, which is jointly therapeutically effective against a tumor disease, of combination partner (a) and combination partner (b) which are administered concurrently but separately, or administered sequentially.

The pharmaceutical compositions for separate administration of the combination partners, or for the administration in a fixed combination, i.e. a single galenical composition comprising the COMBINATION OF THE INVENTION, may be prepared in a manner known per se and are those suitable for enteral, such as oral or rectal, and parenteral administration to subjects (warm-blooded animals), including humans, comprising a therapeutically effective amount of at least one pharmacologically active combination partner alone, e.g. as indicated above, or in combination with one or more pharmaceutically acceptable carriers, especially suitable for enteral or parenteral application.

The novel pharmaceutical composition contains may contain, from about 0.1% to about 99.9%, preferably from about 1% to about 60%, of the active ingredient(s).

Pharmaceutical preparations or compositions for the combination therapy, including fixed combinations or non-fixed combinations, for enteral or parenteral administration are, for example, those in unit dosage forms, such as sugar-coated tablets, tablets, capsules or suppositories, or ampoules. If not indicated otherwise, these are prepared in a manner known per se, for example by means of various conventional mixing, comminution, granulating, sugar-coating, dissolving, lyophilizing processes, or fabrication techniques readily apparent to those skilled in the art. It will be appreciated that the unit content of a combination partner contained in an individual dose of each dosage form need not in itself constitute an effective amount since the necessary effective amount may be reached by administration of a plurality of dosage units.

A unit dosage form containing the combination of agents or individual agents of the combination of agents may be in the form of micro-tablets enclosed inside a capsule, e.g. a gelatin capsule. For this, a gelatin capsule as is employed in pharmaceutical formulations can be used, such as the hard gelatin capsule known as CAPSUGEL™, available from Pfizer.

The unit dosage forms of the present invention may optionally further comprise additional conventional carriers or excipients used for pharmaceuticals. Examples of such carriers include, but are not limited to, disintegrants, binders, lubricants, glidants, stabilizers, and fillers, diluents, colorants, flavours and preservatives. One of ordinary skill in the art may select one or more of the aforementioned carriers with respect to the particular desired properties of the dosage form by routine experimentation and without any undue burden. The amount of each carriers used may vary within ranges conventional in the art. The following references which are all hereby incorporated by reference disclose techniques and excipients used to formulate oral dosage forms. See The Handbook of Pharmaceutical Excipients, 4^{th} edition, Rowe et al., Eds., American Pharmaceuticals Association (2003); and Remington: the Science and Practice of Pharmacy, 20^{th} edition, Gennaro, Ed., Lippincott Williams & Wilkins (2003).

These optional additional conventional carriers may be incorporated into the oral dosage form either by incorporating the one or more conventional carriers into the initial mixture before or during melt granulation or by combining the one or more conventional carriers with the granules in the oral dosage form. In the latter embodiment, the combined mixture may be further blended, e.g., through a V-blender, and subsequently compressed or molded into a tablet, for example a monolithic tablet, encapsulated by a capsule, or filled into a sachet.

Examples of pharmaceutically acceptable disintegrants include, but are not limited to, starches; clays; celluloses; alginates; gums; cross-linked polymers, e.g., cross-linked polyvinyl pyrrolidone or crospovidone, e.g., POLYPLASDONE XL™ from International Specialty Products (Wayne, N.J.); cross-linked sodium carboxymethylcellulose or croscarmellose sodium, e.g., AC-DI-SOL™ from FMC; and cross-linked calcium carboxymethylcellulose; soy polysaccharides; and guar gum. The disintegrant may be present in an amount from about 0% to about 10% by weight of the composition. In one embodiment, the disintegrant is present in an amount from about 0.1% to about 5% by weight of composition.

Examples of pharmaceutically acceptable binders include, but are not limited to, starches; celluloses and derivatives thereof, for example, microcrystalline cellulose, e.g., AVICEL PH™ from FMC (Philadelphia, Pa.), hydroxypropyl cellulose hydroxylethyl cellulose and hydroxylpropylmethyl cellulose METHOCEL™ from Dow Chemical Corp. (Midland, Mich.); sucrose; dextrose; corn syrup; polysaccharides; and gelatin. The binder may be present in an amount from about 0% to about 50%, e.g., 2-20% by weight of the composition.

Examples of pharmaceutically acceptable lubricants and pharmaceutically acceptable glidants include, but are not limited to, colloidal silica, magnesium trisilicate, starches, talc, tribasic calcium phosphate, magnesium stearate, aluminum stearate, calcium stearate, magnesium carbonate, magnesium oxide, polyethylene glycol, powdered cellulose and microcrystalline cellulose. The lubricant may be present in an amount from about 0% to about 10% by weight of the composition. In one embodiment, the lubricant may be present in an amount from about 0.1% to about 1.5% by weight of composition. The glidant may be present in an amount from about 0.1% to about 10% by weight.

Examples of pharmaceutically acceptable fillers and pharmaceutically acceptable diluents include, but are not limited to, confectioner's sugar, compressible sugar, dextrates, dextrin, dextrose, lactose, mannitol, microcrystalline cellulose, powdered cellulose, sorbitol, sucrose and talc. The filler and/or diluent, e.g., may be present in an amount from about 0% to about 80% by weight of the composition.

The combination partners (a) and (b) of the present invention (for example, phosphatidylinositol 3-kinase inhibitor selected from the group consisting of the compound of formula (I), e.g., COMPOUND A or B, or compound of formula (II), e.g., COMPOUND C, and a 17α-Hydroxylase/C_{17,20}-lyase inhibitor, e.g., abiraterone acetate) may be present in the combinations, pharmaceutical compositions and dosage forms disclosed herein in a ratio in the range of 1:1 to 1:100, more preferably 1:20.

In a preferred embodiment, the combination partners COMPOUND A or a pharmaceutically acceptable salt thereof and abiraterone acetate or a pharmaceutically acceptable salt thereof are present in a dosage ratio in the range of 1:1 to 1:3 daily, more preferably 1:1.25, 1:1.5 or 1:1.25 daily.

In a preferred embodiment, the combination partners COMPOUND C or a pharmaceutically acceptable salt thereof and abiraterone acetate or a pharmaceutically acceptable salt thereof are present in a dosage ratio in the range of 1:5 to 1:20 daily, more preferably 1:10, 1:12.5, or 1:16.6 daily.

The combination partners (a) and (b) of the present invention (for example, phosphatidylinositol 3-kinase inhibitor selected from the group consisting of the compound of formula (I), e.g., COMPOUND A or B, or compound of formula (II), e.g., COMPOUND C, and a 17α-Hydroxylase/C_{17,20}-lyase inhibitor, e.g., 1-(2-Chloro-pyridin-4-yl)-3-(4-methyl-pyridin-3-yl)-imidazolidin-2-one) may be present in the combinations, pharmaceutical compositions and dosage forms disclosed herein in a ratio in the range of 1:1 to 1:100, more preferably 1:20.

In a preferred embodiment, the combination partners COMPOUND A or a pharmaceutically acceptable salt thereof and 1-(2-Chloro-pyridin-4-yl)-3-(4-methyl-pyridin-3-yl)-imidazolidin-2-one or a pharmaceutically acceptable salt thereof are present in a dosage ratio in the range of 1:1 to 1:3 daily, more preferably 1:1.25, 1:1.5 or 1:1.25 daily.

In a preferred embodiment, the combination partners COMPOUND C or a pharmaceutically acceptable salt thereof and 1-(2-Chloro-pyridin-4-yl)-3-(4-methyl-pyridin-3-yl)-imidazolidin-2-one or a pharmaceutically acceptable salt thereof are present in a dosage ratio in the range of 1:5 to 1:20 daily, more preferably 1:10, 1:12.5, or 1:16.6 daily.

The optimum ratios, individual and combined dosages, and concentrations of the drug compounds that yield efficacy without toxicity are based on the kinetics of the active ingredients' availability to target sites, and are determined using methods known to those of skill in the art.

In accordance with the present invention, a therapeutically effective amount of each of the combination partners of the combination of the invention may be administered simultaneously or sequentially and in any order, and the components may be administered separately or as a fixed combination. For example, the method of treating a tumor disease according to the invention may comprise (i) administration of the first agent (a) in free or pharmaceutically acceptable salt form, and (ii) administration of an agent (b) in free or pharmaceutically acceptable salt form, simultaneously or sequentially in any order, in jointly therapeutically effective amounts, preferably in synergistically effective amounts, e.g. in daily or intermittently dosages corresponding to the amounts described herein. The individual combination partners of the COMBINATION OF THE INVENTION may be administered separately at different times during the course of therapy or concurrently in divided or single combination forms. Furthermore, the term “administering” also encompasses the use of a pro-drug of a combination partner that convert in vivo to the combination partner as such. The instant invention is therefore to be understood as embracing all such regimens of simultaneous or alternating treatment and the term “administering” is to be interpreted accordingly.

The effective dosage of each of the combination partners employed in the COMBINATION OF THE INVENTION may vary depending on the particular compound or pharmaceutical composition employed, the mode of administration, the condition being treated, and the severity of the condition being treated. Thus, the dosage regimen of the combination of the invention is selected in accordance with a variety of factors including the route of administration and the renal and hepatic function of the patient. A clinician or physician of ordinary skill can readily determine and prescribe the effective amount of the single active ingredients required to alleviate, counter or arrest the progress of the condition.

The effective dosage of each of the combination partners may require more frequent administration of one of the compound(s) as compared to the other compound(s) in the combination. Therefore, to permit appropriate dosing, packaged pharmaceutical products may contain one or more dosage forms that contain the combination of compounds, and one or more dosage forms that contain one of the combination of compounds, but not the other compound(s) of the combination.

When the combination partners, which are employed in the COMBINATION OF THE INVENTION, are applied in the form as marketed as single drugs, their dosage and mode of administration can be in accordance with the information provided on the package insert of the respective marketed drug, if not mentioned herein otherwise.

The dose of a compound of the formula I, especially COMPOUND A, or a pharmaceutically acceptable salt thereof to be administered to warm-blooded animals, for example humans of approximately 70 kg body weight, is preferably from approximately 3 mg to approximately 5 g, more preferably from approximately 10 mg to approximately 1.5 g, more preferably from approximately 100 mg to about 1200 mg, most preferably from about 100 mg to about 1000 mg per person per day, divided preferably into 1 to 3 single doses which may, for example, be of the same size.

The compound of formula II, especially COMPOUND C, is preferably administered daily at a dose in the range of from about 0.001 to 1000 mg/kg body weight daily and more preferred from 1.0 to 30 mg/kg body weight. In one preferred embodiment, the dosage compound of formula I, especially COMPOUND C, is in the range of about 10 mg to about 2000 mg/day, especially if the warm-blooded animal is an adult human.

The 17α-Hydroxylase/C_{17,20}-lyase inhibitor or pharmaceutically acceptable salt thereof, particularly abiraterone acetate, is preferably administered at a dose in the range of from about 0.01 mg/kg body weight daily to about 100 mg/kg body weight daily. In one preferred embodiment, the dosage of abiraterone acetate is in the range of about 750 to 1000 mg/day for an adult human of approximately 70 kg body weight, divided preferably into 1 to 4 single doses which may, for example, be of the same size.

The 17α-Hydroxylase/C_{17,20}-lyase inhibitor or pharmaceutically acceptable salt thereof, particularly 1-(2-Chloro-pyridin-4-yl)-3-(4-methyl-pyridin-3-yl)-imidazolidin-2-one, is preferably administered at a dose in the range of from about 0.01 mg/kg body weight daily to about 300 mg/kg body weight daily. In one preferred embodiment, the dosage of 1-(2-Chloro-pyridin-4-yl)-3-(4-methyl-pyridin-3-yl)-imidazolidin-2-one is in the range of about 10 to 1000 mg/day for an adult human of approximately 70 kg body weight, divided preferably into 1 to 4 single doses which may, for example, be of the same size.

The optimal dosage of each combination partner for treatment of a tumor disease can be determined empirically for each individual using known methods and will depend upon a variety of factors, including, though not limited to, the degree of advancement of the disease; the age, body weight, general health, gender and diet of the individual; the time and route of administration; and other medications the individual is taking. Optimal dosages may be established using routine testing and procedures that are well known in the art.

The amount of each combination partner that may be combined with the carrier materials to produce a single dosage form will vary depending upon the individual treated and the particular mode of administration. In some embodiments the unit dosage forms containing the combination of agents as described herein will contain the amounts of each agent of the combination that are typically administered when the agents are administered alone.

Frequency of dosage may vary depending on the compound used and the particular condition to be treated. In general, the use of the minimum dosage that is sufficient to provide effective therapy is preferred. Patients may generally be monitored for therapeutic effectiveness using assays suitable for the condition being treated, which will be familiar to those of ordinary skill in the art.

The COMBINATION OF THE INVENTION is useful for the inhibition the growth of tumors. In one preferred embodiment, the disease to be treated with a COMBINATION OF THE INVENTION is a prostate cancer.

The present invention relates to a method of treating a tumor disease comprising administering to subject in need thereof a COMBINATION OF THE INVENTION in a quantity, which is jointly therapeutically effective against said tumor disease. In particular, the tumor disease to be treated with a COMBINATION OF THE INVENTION is a prostate cancer, preferably castration resistant prostate cancer.

Moreover, the present invention also provides a method of inhibiting the formation of metastases in a subject having tumor disease, in particular a prostate cancer, comprising administering to a subject in need thereof an amount of a COMBINATION OF THE INVENTION in a quantity which is therapeutically effective against said tumor disease.

Moreover, the present invention also pertains to the use of a COMBINATION OF THE INVENTION for the treatment of a tumor disease, in particular a prostate cancer, and for the preparation of a medicament for the treatment of a tumor disease.

The present invention further pertains to the use of a phosphatidylinositol 3-kinase inhibitor selected from the group consisting of the compound of formula (I), e.g., COMPOUND A or B, or compound of formula (II), e.g., COMPOUND C, or a pharmaceutically acceptable salt thereof in combination with a 17α-Hydroxylase/C_{17,20}-lyase inhibitor or pharmaceutically acceptable salt thereof for the preparation of a medicament for the treatment of a tumor disease, in particular prostate cancer.

In a further aspect of the present invention, the COMBINATION OF THE INVENTION is further administered with at least one additional therapeutic agent for use in the treatment of a tumor disease or for use inhibiting the formation of metastases in a subject having a tumor disease. Suitable additional therapeutic agents which may be administered with the COMBINATION OF THE INVENTION include, but are not limited to, steroids, such as corticosteroids or glucocorticoids. Examples of suitable steroids include, but not limited to, hydrocortisone, dexamethasone, prednisolone, prednisone and combinations thereof, and any pharmaceutically acceptable salt thereof.

Preferably, the steroid is prednisone or a pharmaceutically acceptable salt thereof. Prednisone (CAS Registry no. 53-03-2) is commercially available and having formula (IV)

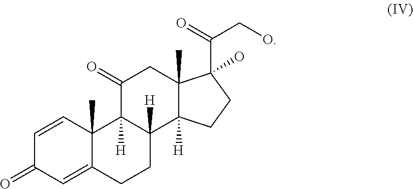

In one embodiment, the present invention provides a combination comprising (a) a phosphatidylinositol 3-kinase inhibitor selected from the group consisting of the compound of formula (I) or the compound of formula (II) or a pharmaceutically acceptable salt thereof, (b) a 17α-Hydroxylase/C_{17,20}-lyase inhibitor or a pharmaceutically acceptable salt thereof, and (c) at least one additional therapeutic agent, for simultaneous, separate or sequential use. Preferably, the 17α-Hydroxylase/C_{17,20}-lyase inhibitor is a steroid. More preferably, the 17α-Hydroxylase/C_{17,20}-lyase inhibitor is abiraterone acetate or a pharmaceutically acceptable salt thereof.

In a further embodiment, the present invention comprises a combination comprising (a) COMPOUND A or a pharmaceutically acceptable salt thereof, (b) abiraterone acetate or a pharmaceutically acceptable salt thereof, and (c) prednisone or a pharmaceutically acceptable salt thereof.

In a further embodiment, the present invention comprises a combination comprising (a) COMPOUND C or a pharmaceutically acceptable salt thereof, (b) abiraterone acetate or a pharmaceutically acceptable salt thereof, and (c) prednisone or a pharmaceutically acceptable salt thereof.

In one embodiment, the present invention provides a combination comprising (a) a phosphatidylinositol 3-kinase inhibitor selected from the group consisting of the compound of formula (I) or the compound of formula (II) or a pharmaceutically acceptable salt thereof, (b) a 17α-Hydroxylase/C_{17,20}-lyase inhibitor or a pharmaceutically acceptable salt thereof, and (c) at least one additional therapeutic agent, for simultaneous, separate or sequential use. Preferably, the 17α-Hydroxylase/C_{17,20}-lyase inhibitor is a steroid. More preferably, the 17α-Hydroxylase/C_{17,20}-lyase inhibitor is 1-(2-Chloro-pyridin-4-yl)-3-(4-methyl-pyridin-3-yl)-imidazolidin-2-one or a pharmaceutically acceptable salt thereof.

In a further embodiment, the present invention comprises a combination comprising (a) COMPOUND A or a pharmaceutically acceptable salt thereof, (b) 1-(2-Chloro-pyridin-4-yl)-3-(4-methyl-pyridin-3-yl)-imidazolidin-2-one or a pharmaceutically acceptable salt thereof, and (c) prednisone or a pharmaceutically acceptable salt thereof.

In a further embodiment, the present invention comprises a combination comprising (a) COMPOUND C or a pharmaceutically acceptable salt thereof, (b) 1-(2-Chloro-pyridin-4-yl)-3-(4-methyl-pyridin-3-yl)-imidazolidin-2-one or a pharmaceutically acceptable salt thereof, and (c) prednisone or a pharmaceutically acceptable salt thereof.

Steroids, particularly prednisone, may be administered in a dosage range of about 0.5 mg to about 20 mg/day, divided into 1 to 3 single doses. In a preferred embodiment, prednisone is administered at a dosage of 10 mg/day, divided into 2 single doses. In a preferred embodiment, prednisone is administered at a dosage of 5 mg two times per day (5 m.g. B.I.D.).

The combination partners (a) a phosphatidylinositol 3-kinase inhibitor selected from the group consisting of the compound of formula (I) or the compound of formula (II) or a pharmaceutically acceptable salt thereof, (b) a 17α-Hydroxylase/C_{17,20}-lyase inhibitor, particularly abiraterone acetate, or a pharmaceutically acceptable salt thereof, and (c) at least one additional therapeutic agent of the present invention may be present in the combinations, pharmaceutical compositions and dosage forms disclosed herein in a ratio in the range of 1:100:1 to 100:100:1, more preferably 6:100:1 to 80:100:1. In the preferred embodiment, the additional therapeutic agent is prednisone.

In a preferred embodiment, the combination partners COMPOUND A or a pharmaceutically acceptable salt thereof, abiraterone acetate or a pharmaceutically acceptable salt thereof and prednisone or a pharmaceutically acceptable salt thereof are present in a dosage ratio in the range of 40:100:1 to 80:100:1, 40:100:1, 60:100:1 or 80:100:1 daily.

In a preferred embodiment, the combination partners COMPOUND C or a pharmaceutically acceptable salt thereof, abiraterone acetate or a pharmaceutically acceptable salt thereof and prednisone or a pharmaceutically acceptable salt thereof are present in a dosage ratio in the range of 6:100:1 to 10:100:1, 6:100:1, 8:100:1, or 10:100:1 daily.

The present invention further comprises a pharmaceutical composition comprising (a) a phosphatidylinositol 3-kinase inhibitor selected from the group consisting of the compound of formula (I) or the compound of formula (II) or a pharmaceutically acceptable salt thereof, (b) a 17α-Hydroxylase/C_{17,20}-lyase inhibitor, particularly abiraterone acetate, or a pharmaceutically acceptable salt thereof, and (c) at least one additional therapeutic agent, for use in the treatment of a tumor disease.

The combination partners (a) a phosphatidylinositol 3-kinase inhibitor selected from the group consisting of the compound of formula (I) or the compound of formula (II) or a pharmaceutically acceptable salt thereof, (b) a 17α-Hydroxylase/C_{17,20}-lyase inhibitor, particularly 1-(2-Chloro-pyridin-4-yl)-3-(4-methyl-pyridin-3-yl)-imidazolidin-2-one, or a pharmaceutically acceptable salt thereof, and (c) at least one additional therapeutic agent of the present invention may be present in the combinations, pharmaceutical compositions and dosage forms disclosed herein in a ratio in the range of 1:100:1 to 100:100:1, more preferably 6:100:1 to 80:100:1. In the preferred embodiment, the additional therapeutic agent is prednisone.

In a preferred embodiment, the combination partners COMPOUND A or a pharmaceutically acceptable salt thereof, 1-(2-Chloro-pyridin-4-yl)-3-(4-methyl-pyridin-3-yl)-imidazolidin-2-one or a pharmaceutically acceptable salt thereof and prednisone or a pharmaceutically acceptable salt thereof are present in a dosage ratio in the range of 40:100:1 to 80:100:1, 40:100:1, 60:100:1 or 80:100:1 daily.

In a preferred embodiment, the combination partners COMPOUND C or a pharmaceutically acceptable salt thereof, 1-(2-Chloro-pyridin-4-yl)-3-(4-methyl-pyridin-3-yl)-imidazolidin-2-one or a pharmaceutically acceptable salt thereof and prednisone or a pharmaceutically acceptable salt thereof are present in a dosage ratio in the range of 6:100:1 to 10:100:1, 6:100:1, 8:100:1, or 10:100:1 daily.

The present invention further comprises a pharmaceutical composition comprising (a) a phosphatidylinositol 3-kinase inhibitor selected from the group consisting of the compound of formula (I) or the compound of formula (II) or a pharmaceutically acceptable salt thereof, (b) a 17α-Hydroxylase/C_{17,20}-lyase inhibitor, particularly 1-(2-Chloro-pyridin-4-yl)-3-(4-methyl-pyridin-3-yl)-imidazolidin-2-one, or a pharmaceutically acceptable salt thereof, and (c) at least one additional therapeutic agent, for use in the treatment of a tumor disease.

In accordance with one aspect of the present invention, the combination comprising (a) a phosphatidylinositol 3-kinase inhibitor selected from the group consisting of the compound of formula (I) or the compound of formula (II) or a pharmaceutically acceptable salt thereof, (b) a 17α-Hydroxylase/C_{17,20}-lyase inhibitor, particularly abiraterone acetate, or a pharmaceutically acceptable salt thereof, and (c) at least one additional therapeutic agent, is useful for the treatment of a tumor disease. Examples of tumor disease which may be treated with this combination includes, but is not limited to, benign or malignant tumors, carcinoma of the brain, kidney, liver, bladder, breast, gastric, ovaries, colon, rectum, prostate, pancreas, lung, vagina or thyroid, or gastrointestinal cancers.

In accordance with one aspect of the present invention, the combination comprising (a) a phosphatidylinositol 3-kinase inhibitor selected from the group consisting of the compound of formula (I) or the compound of formula (II) or a pharmaceutically acceptable salt thereof, (b) a 17α-Hydroxylase/C_{17,20}-lyase inhibitor, particularly 1-(2-Chloro-pyridin-4-yl)-3-(4-methyl-pyridin-3-yl)-imidazolidin-2-one, or a pharmaceutically acceptable salt thereof, and (c) at least one additional therapeutic agent, is useful for the treatment of a tumor disease. Examples of tumor disease which may be treated with this combination includes, but is not limited to, benign or malignant tumors, carcinoma of the brain, kidney, liver, bladder, breast, gastric, ovaries, colon, rectum, prostate, pancreas, lung, vagina or thyroid, or gastrointestinal cancers.

In a preferred embodiment, the tumor disease treated is prostate cancer.

In another preferred embodiment, the tumor disease treated is prostate cancer resistant to treatment with abiraterone acetate or a pharmaceutically acceptable salt thereof. In another preferred embodiment, the tumor treated is castration-resistant prostate cancer which is resistant to treatment with abiraterone acetate or a pharmaceutically acceptable salt thereof.

The present invention further relates to a method of treating a patient having a tumor disease which comprises administering to a patient in need thereof a combination comprising (a) a phosphatidylinositol 3-kinase inhibitor selected from the group consisting of the compound of formula (I) or the compound of formula (II) or a pharmaceutically acceptable salt thereof, (b) a 17α-Hydroxylase/C_{17,20}-lyase inhibitor, particularly abiraterone acetate, or a pharmaceutically acceptable salt thereof, and (c) at least one additional therapeutic agent in a quantity which is jointly therapeutically effective against said tumor disease.

In one embodiment, the additional therapeutic agent is a steroid, such as corticosteroid or glucocorticoid. In a further example, the additional therapeutic agent is a steroid selected from the group consisting of hydrocortisone, dexamethasone, prednisolone, prednisone and combinations thereof, and any pharmaceutically acceptable salt thereof.

In a further embodiment, the present invention relates to a method of treating a patient having a tumor disease which comprises administering to a patient in need thereof a combination comprising (a) COMPOUND A or a pharmaceutically acceptable salt thereof, (b) abiraterone acetate or a pharmaceutically acceptable salt thereof, and (c) prednisone or a pharmaceutically acceptable salt thereof in a quantity which is jointly therapeutically effective against said tumor disease.

In a further embodiment, the present invention relates to a method of treating a patient having a tumor disease which comprises administering to a patient in need thereof a combination comprising (a) COMPOUND C or a pharmaceutically acceptable salt thereof, (b) abiraterone acetate or a pharmaceutically acceptable salt thereof, and (c) prednisone or a pharmaceutically acceptable salt thereof in a quantity which is jointly therapeutically effective against said tumor disease.

The present invention further relates to a method of inhibiting the formation of metastases in a subject having a tumor disease which comprises administering to a patient in need thereof a combination comprising (a) a phosphatidylinositol 3-kinase inhibitor selected from the group consisting of the compound of formula (I) or the compound of formula (II) or a pharmaceutically acceptable salt thereof, (b) a 17α-Hydroxylase/C_{17,20}-lyase inhibitor, particularly abiraterone acetate, or a pharmaceutically acceptable salt thereof, and (c) at least one additional therapeutic agent in a quantity which is jointly therapeutically effective against said tumor disease.

In another preferred embodiment, the tumor disease treated is prostate cancer resistant to treatment with abiraterone acetate or a pharmaceutically acceptable salt thereof. In another preferred embodiment, the tumor treated is castration-resistant prostate cancer which is resistant to treatment with abiraterone acetate or a pharmaceutically acceptable salt thereof.

The present invention further relates to a method of treating a patient having a tumor disease which comprises administering to a patient in need thereof a combination comprising (a) a phosphatidylinositol 3-kinase inhibitor selected from the group consisting of the compound of formula (I) or the compound of formula (II) or a pharmaceutically acceptable salt thereof, (b) a 17α-Hydroxylase/C_{17,20}-lyase inhibitor, particularly 1-(2-Chloro-pyridin-4-yl)-3-(4-methyl-pyridin-3-yl)-imidazolidin-2-one, or a pharmaceutically acceptable salt thereof, and (c) at least one additional therapeutic agent in a quantity which is jointly therapeutically effective against said tumor disease.

In one embodiment, the additional therapeutic agent is a steroid, such as corticosteroid or glucocorticoid. In a further example, the additional therapeutic agent is a steroid selected from the group consisting of hydrocortisone, dexamethasone, prednisolone, prednisone and combinations thereof, and any pharmaceutically acceptable salt thereof.

In a further embodiment, the present invention relates to a method of treating a patient having a tumor disease which comprises administering to a patient in need thereof a combination comprising (a) COMPOUND A or a pharmaceutically acceptable salt thereof, (b) 1-(2-Chloro-pyridin-4-yl)-3-(4-methyl-pyridin-3-yl)-imidazolidin-2-one or a pharmaceutically acceptable salt thereof, and (c) prednisone or a pharmaceutically acceptable salt thereof in a quantity which is jointly therapeutically effective against said tumor disease.

In a further embodiment, the present invention relates to a method of treating a patient having a tumor disease which comprises administering to a patient in need thereof a combination comprising (a) COMPOUND C or a pharmaceutically acceptable salt thereof, (b) 1-(2-Chloro-pyridin-4-yl)-3-(4-methyl-pyridin-3-yl)-imidazolidin-2-one or a pharmaceutically acceptable salt thereof, and (c) prednisone or a pharmaceutically acceptable salt thereof in a quantity which is jointly therapeutically effective against said tumor disease.

The present invention further relates to a method of inhibiting the formation of metastases in a subject having a tumor disease which comprises administering to a patient in need thereof a combination comprising (a) a phosphatidylinositol 3-kinase inhibitor selected from the group consisting of the compound of formula (I) or the compound of formula (II) or a pharmaceutically acceptable salt thereof, (b) a 17α-Hydroxylase/C_{17,20}-lyase inhibitor, particularly 1-(2-Chloro-pyridin-4-yl)-3-(4-methyl-pyridin-3-yl)-imidazolidin-2-one, or a pharmaceutically acceptable salt thereof, and (c) at least one additional therapeutic agent in a quantity which is jointly therapeutically effective against said tumor disease.

In one embodiment, the additional therapeutic agent is a steroid, such as corticosteroid or glucocorticoid. In a further example, the additional therapeutic agent is a steroid steroids selected from the group consisting of hydrocortisone, dexamethasone, prednisolone, prednisone and combinations thereof, and any pharmaceutically acceptable salt thereof.

Moreover, the present invention provides a commercial package comprising as active ingredients COMBINATION OF THE INVENTION, together with instructions for the simultaneous, separate or sequential use thereof in the treatment of a tumor disease, particularly prostate cancer.

The structure of the active ingredients identified by code numbers, generic or trade names may be taken from the actual edition of the standard compendium “The Merck Index” or from databases, e.g., Patents International (e.g, IMS World Publications). The corresponding content thereof is hereby incorporated by reference.

The following Examples illustrate the invention described above; they are not, however, intended to limit the scope of the invention in any way. The beneficial effects of the pharmaceutical combination of the present invention can also be determined by other test models known as such to the person skilled in the pertinent art.

Utility of the COMBINATION OF THE PRESENT INVENTION, as described herein, may be demonstrated in vitro, in animal test methods as well as in clinical studies. For example in the utility of the compounds of formula (I) in accordance with the present invention may be demonstrated in accordance with the methods hereinafter described:

### Example 1

### Clinical Trial

A clinical study using (a) a phosphatidylinositol 3-kinase inhibitor that is either COMPOUND A or its monotosylate salt or COMPOUND C or its hydrochloride salt, in combination with (b) abiraterone acetate or a pharmaceutically acceptable salt thereof, and (c) prednisone for treatment of patients with castration-resistant prostate cancer after failure of abiraterone acetate therapy is investigated.

An open-label, unblinded study of the combination comprising (a) either COMPOUND A or its monotosylate salt or COMPOUND C or its hydrochloride salt, (b) abiraterone acetate or a pharmaceutically acceptable salt thereof, and (c) prednisone is conducted in patients who are diagnosed with a castration-resistant prostate cancer and after failure of abiraterone acetate therapy. In the first phase, a dose-escalation study is conducted to determine the maximal tolerated dose (MTD) and/or recommended dose for expansion (RDE) of both (a) COMPOUND A or its monotosylate salt in combination with abiraterone acetate and prednisone (hereinafter referred to as “COMPOUND A Arm 1”), and (b) COMPOUND C or its monohydrochloride salt in combination with abiraterone acetate and prednisone (hereinafter referred to as “COMPOUND C Arm 1”) in patients diagnosed with castration-resistant prostate cancer after failure of abiraterone acetate therapy. Approximately 15 to 25 patients are enrolled in the COMPOUND A Arm 1, and approximately 10 to 20 patients are enrolled in the COMPOUND C Arm 1 in the first phase. In the second phase, a dose expansion phase is conducted to investigate the antitumor activity of both (a) COMPOUND A or its monotosylate salt in combination with abiraterone acetate and prednisone (hereinafter referred to as “COMPOUND A Arm 2”), and (b) COMPOUND C or its monohydrochloride salt in combination with abiraterone acetate and prednisone (hereinafter referred to as “COMPOUND C Arm 2”) in patients diagnosed with castration-resistant prostate cancer after failure of treatment with abiraterone acetate therapy. Approximately 35 to 45 patients are enrolled in the COMPOUND A Arm 2, and approximately 35 to 45 patients are enrolled in the COMPOUND C Arm 2 in the second phase. Patients are treated until disease progression, unacceptable toxicity, death, or discontinuation from the study treatment due to any other reason.

For the clinical study, “failure of abiraterone acetate therapy” is defined as progression of the castration-resistant prostate cancer after treatment with abiraterone acetate therapy alone or abiraterone acetate therapy in combination with prednisone or corticosteroids administered by topical application, inhalation, eye drops, or local injection. In the first phase, patients have a documented progressive disease to prior abiraterone acetate therapy according to Prostate Cancer Working Group 2 (PCWG2) criteria according to at least one of the following criteria: (a) patient who progressed based solely upon rising levels of the serum marker Prostate-Specific Antigen (PSA), wherein the sequence of rising values occurs on three (3) consecutive occasions of at least 1 week intervals and having 5.0 ng/mL minimum level for entry into the study, (b) patient who manifest disease progression per Response Evaluation Criteria in Solid Tumors (“RECIST”) independent of PSA level, and/or (c) patient with bone only progression according to PCWG2. In the second phase, patients must have had a prior Prostate-Specific Antigen response followed by documented Prostate-Specific Antigen (“PSA”) progression on abiraterone acetate monotherapy. “PSA progression” is defined as a sequence of rising levels of PSA on three (3) consecutive occasions of at least 1 week intervals and having 5.0 ng/mL minimum level for entry into the study,

Potential patients are screened within 7 to 28 day days prior to treatment start. The following inclusion and exclusion criteria define those patients eligible for the study:

Inclusion Criteria:

Patients eligible for this study are those satisfying all of the following criteria:
Adult males ≧18 years old
Eastern Cooperative Oncology Group Performance Status 2
Patient having a castrate level of testosterone (<=50 ng/dL or 1.7 nmol/L). (Castrate status must be maintained by continued Gonadotropin-releasing hormone (GnRH) analogues unless patient has undergone surgical orchiectomy).
Histologically or cytologically confirmed diagnosis of advanced or metastatic castration resistant prostate cancer.
Advanced or metastatic castration-resistant prostate cancer progression after abiraterone acetate failure
Patients having no more than 2 lines of prior chemotherapies including cytotoxic agents (i.e. docetaxel)
Discontinuation of all anti-androgen, anti-neoplastic or investigational treatment >=4 weeks (6 weeks for bicalutamide). Patient has adequate bone marrow and organ function as defined by the following laboratory values:

Additional inclusion criteria for First Phase (dose escalation part) only):
Patient having documented progressive disease to the previous line of therapy according to PCWG2 criteria:
   Patient who progressed based solely on Prostate-Specific Antigen (PSA) rising levels, should have had a sequence of rising values on 3 consecutive occasions of at least 1 week intervals and should have 5.0 ng/mL minimum level for entry.
   Patients who manifested disease progression per RECIST are eligible independent of PSA.
   Patients with bone only progression according to PCWG2.

Additional inclusion criteria for Second Phase (dose expansion part) only:
Patient having a prior Prostate-Specific Antigen (PSA) response, followed by documented PSA progression on abiraterone acetate therapy.
“PSA progression” is defined as a sequence of rising values on 3 consecutive occasions of at least 1 week intervals and should have 5.0 ng/mL minimum level for entry.
Abiraterone Acetate is the last treatment prior to enrolment in the dose expansion part of the study and no other anticancer therapy is allowed between Abiraterone Acetate and study entry.

Exclusion Criteria:

Patients eligible for this study are those that do not meet any of the following criteria:
Previous treatment with PI3K pathway inhibitors (e.g. PI3K, AKT, mTOR inhibitor), ketoconazole, or other CYP17 inhibitors (exception of AA), or MDV3100.
Patient having active uncontrolled or symptomatic Central Nervous System (CNS) metastases. A patient with controlled and asymptomatic CNS metastases may participate in this trial. As such, the patient must have completed any prior treatment for CNS metastases >28 days (including radiotherapy and/or surgery) prior to start of treatment in this study and should not be receiving chronic corticosteroid therapy for the CNS metastases.
Inadequately controlled hypertension (e.g. systolic blood pressure >=160 mmHg or diastolic blood pressure >=95 mmHg)
Patient having a QT interval corrected for heart rate using Fridericia's formula (QTcF) >480 msec on the screening Electrocardiogram (using the QT interval corrected for heart rate using Fridericia's formula), has a short/long QT syndrome, or history of QT prolongation/Torsades de Pointes
Patient is currently receiving treatment with medication that has a known risk to prolong the QT interval or inducing Torsades de Pointes, and the treatment cannot be discontinued or switched to a different medication prior to treatment start.
Patient is currently receiving increasing or chronic treatment (>5 days) with corticosteroids or another immunosuppressive agent, as chronic administration of corticosteroids (>5 days) can induce CYP3A4. The following corticosteroids are permitted:
a. Prednisone 5 mg BID as part of the study treatment;
b. Topical applications (e.g., rash), inhaled sprays (e.g., obstructive airways diseases), eye drops or local injections (e.g., intra-articular)
Patient having impairment of gastrointestinal (GI) function or GI disease that may significantly alter the absorption of study drugs (e.g., ulcerative diseases, uncontrolled nausea, vomiting, diarrhea, malabsorption syndrome, or small bowel resection)
Patient having a medically documented history of or active major depressive episode, bipolar disorder (I or II), obsessive-compulsive disorder, schizophrenia, a history of suicidal attempt or ideation, or homicidal ideation (e.g. risk of doing harm to self or others) and/or (a) patient has ≦CTCAE grade 3 anxiety, (b) Patient has a Generalized Anxiety Disorder 7-item scale (GAD-7) mood scale score ≧15, or (c) Patient has a score ≧12 on the Patient Health Questionaire-9 item (PHQ-9) questionnaire.
Patients who has experienced dose reductions and/or treatment interruptions due to abiraterone acetate related toxicities (i.e. serious adverse events, adverse events, liver toxicities during abiraterone acetate treatment

Additional exclusion criteria for Second Phase (dose expansion part) only:
Patients who has received any anticancer systemic therapy between abiraterone acetate and study entry
   During the screening, the patient's diagnosis and extent of cancer (including staging, histology/cytology and sites of disease at study entry, PSA history and PSA level), demography, medical history, prior antineoplastic therapies, all medications and significant non-drug therapies taken within 28 days before first dose, and other physical characteristics (e.g, vital signs, height, weight, physical examination, ECOG, laboratory evaluations of blood, ECG, cardiac imaging, CT/MRI and bone scan assessments of tumors and additional radiological assessments if clinically indicated, and mood) are assessed.

In the First Phase (Dose Escalation) of the clinical trial, the primary objective is to determine (a) the MTD and/or RDE of COMPOUND A or its monotosylate salt in combination with abiraterone acetate and (b) the MTD and/or RDE of COMPOUND C or its monohydrochloride salt in combination with abiraterone acetate. A secondary objective is to determine the percent change from baseline in PSA at Week 12 as well as the best percentage change from baseline at any time as calculated using water fall plots. The proportion of patients with PSA decrease from baseline of at least 30% at Week 12 or later is calculated for patients of the first phase.

In the COMPOUND A Arm 1 group, patients are provisionally administered each of the following: (a) 200 mg of COMPOUND A, per oral, twice daily (B.I.D.) for a total daily dose of 400 mg; (b) 1000 mg abiraterone acetate, per oral, once daily (Q.D.); and (c) 5 mg prednisone, per oral, twice daily (B.I.D.) for a total daily dose of 10 mg. In the COMPOUND C Arm 1 group, patients are provisionally administered each of the following: (a) 60 mg of COMPOUND C, per oral, once daily (Q.D.), (b) 1000 mg abiraterone acetate, per oral, once daily (Q.D.); and (c) 5 mg prednisone, per oral, twice daily (B.I.D.) for a total daily dose of 10 mg. COMPOUND A may be administered in the form of its free base or its monotosylate salt form. A complete treatment cycle is defined as 35 calendar days consisting of (a) a 7-day run-in period during which abiraterone acetate or its pharmaceutically acceptable salt is given once daily and prednisone is given twice daily and (b) 28 days combination treatment period during which COMPOUND A or its monotosylate salt and prednisone are given twice daily and abiraterone acetate or its pharmaceutically acceptable salt is given once daily. Combination treatment with COMPOUND A or its monotosylate salt begins on Day 8 of Cycle 1. The last day of the first treatment cycle is Day 35. All subsequent treatment cycles for COMPOUND A Arm 1 consist of 28 calendar days during which COMPOUND A or its monotosylate salt and prednisone are given twice daily and abiraterone acetate is given once daily.

In the COMPOUND C Arm 1 group, patients are provisionally administered each of the following: (a) 60 mg of COMPOUND C, per oral, once daily (Q.D.); (b) 1000 mg abiraterone acetate, per oral, once daily (Q.D.); and (c) 5 mg prednisone, per oral, twice daily (B.I.D.) for a total daily dose of 10 mg. COMPOUND C may be administered in the form of its free base or its monohydrochloride salt form. A complete treatment cycle consists of 28 calendar days during which COMPOUND C or its monohydrochloride salt and abiraterone acetate are given once daily and prednisone is given twice daily. The last day of the complete treatment cycle is Day 28.

A cohort of approximately 3-6 patients is treated at the next dose level. Each cohort is consisting of newly enrolled patients. To account for the potential of drop-outs during the first cycle of treatment (e.g., due to early disease progression), up to up to 2 additional patients may be enrolled, if these additional patients can be treated within 14 days after the third patient is first dosed with the COMPOUND A or its monotosylate salt. The dose levels for the dose-escalation phase of the clinical study are defined as follows for the COMPOUND A Arm 1 Group:

| | COMPOUND | Abiraterone | |
|---|---|---|---|
| Dose level | A | acetate | Prednisone |
| Dose | 200 mg BID | 1000 mg (QD) | 5 mg BID (2 × 5 mg for a |
| Level 1 | (2 × 200 mg | | total of 10 mg a day) |
| | for total of | | |
| | 400 mg a | | |
| | day) | | |
| Dose | 300 mg BID | 1000 mg (QD) | 5 mg BID (2 × 5 mg for a |
| Level 2 | (2 × 300 | | total of 10 mg a day) |
| | mg for total | | |
| | of 600 mg a | | |
| | day) | | |
| Dose | 400 mg BID | 1000 mg (QD) | 5 mg BID (2 × 5 mg for a |
| Level 3 | (2 × 400 | | total of 10 mg a day) |
| | mg for total | | |
| | of 800 mg a | | |
| | day) | | |

Further, the dose levels for the dose-escalation phase of the clinical study are defined as follows for the COMPOUND C Arm 1 Group:

| | COMPOUND | Abiraterone | |
|---|---|---|---|
| Dose level | C | acetate | Prednisone |
| Dose | 60 mg (QD) | 1000 mg (QD) | 5 mg BID (2 × 5 mg for a |
| Level 1 | | | total of 10 mg a day) |
| Dose | 80 mg (QD) | 1000 mg (QD) | 5 mg BID (2 × 5 mg for a |
| Level 2 | | | total of 10 mg a day) |
| Dose | 100 mg (QD) | 1000 mg (QD) | 5 mg BID (2 × 5 mg for a |
| Level 3 | | | total of 10 mg a day) |

In the first phase, the individual dose is dependent on the current dose level at which the patient enters the study. A five-parameter adaptive Bayesian logistic regression model guided by escalation with overdose control (EWOC) principle is used for dose level selection and for determination of the MTD and/or RDE of the COMPOUND A combination treatment and the COMPOUND C combination treatment. Patients are considered evaluable for the dose-determining set if the patient experiences a dose-limiting toxicity (DLT) during cycle 1, or meets the minimum treatment exposure and safety evaluations required as defined by the following criteria:
(a) Patients in the COMPOUND A Arm 1 group: The minimum exposure criterion is at least 21 of the 28 full daily planned doses of abiraterone acetate and COMPOUND A or its monotosylate salt between Day 8 and Day 35 of Cycle 1.
(b) Patients in the COMPOUND C Arm 1 group: The minimum exposure criterion is at least 21 of the 28 full daily planned doses of abiraterone acetate and COMPOUND C or its monohydrochloride salt between Day 1 and Day 28 of Cycle 1.
(c) Patients should have completed the required safety evaluations during Cycle 1 to an extent that the investigator or physician can determine that no unacceptable toxicities occurred in Cycle 1 for this dosing cohort.

To determine the dose level for the next cohort in the dose-escalation, the available toxicity information (including dose limiting toxicities and adverse events that are not dose limiting toxicities), pharmacokinetics, pharmacodynamics, efficacy information and recommendations from the five-parameter adaptive Bayesian logistic regression model (BLRM) are evaluated. A 5 parameter BLRM for each combination treatment is fitted on Cycle 1 dose-limiting toxciity data (i.e., absence or presence of DLT) accumulated throughout the dose-escalation to model the dose-toxicity relationship of abiraterone acetate and either COMPOUND A or COMPOUND C when given in combination. The dose for the next cohort is not exceeding the maximum dose allowed by the five-parameter adaptive Bayesian logistic regression model. After completion of cycle 1 of each cohort, the BLRM is used to make recommendations about the next dose level, with the following exceptions:
If 2 of the first 3 patients in a cohort experience a dose limiting toxicity (DLT), further enrollment to that cohort is suspended. The BLRM is updated with this new information before additional patients are enrolled to the study.
If a decision is made to escalate to a higher dose level, but one or more additional patient(s) are treated at the preceding dose level experiences a dose limiting toxicity (DLT), in cycle 1, then the BRLM is updated before any additional patient is enrolled to the higher dose level.
   Dose escalation is continued until identification of the MTD and/or RDE.

A dose-limiting toxicity (DLT) is defined as an adverse event or abnormal laboratory value assessed as unrelated to disease, disease progression, inter-current illness, or concomitant medications that occurs within the first cycle and meets any of the criteria included below. National Cancer Institute Common Terminology Criteria for Adverse events (NCI CTCAE) version 4.03, which is hereby incorporated by reference in its entirety, will be used for all grading. For the purpose of dose-escalation decisions, the following DLTs are considered for these drug combinations:

TABLE 1

| TOXICITY | ANY OF THE FOLLOWING CRITERIA |
|---|---|
| Hematologic | CTCAE grade 4 neutropenia |
| | CTCAE grade 4 thrombocytopenia |
| | Febrile neutropenia (ANC <1.0 × 10⁹/L, fever ≧38.3° C.) |
| | CTCAE Grade ≧3. |
| Renal | ≧CTCAE grade 3 serum creatinine |
| Hepatic | Total bilirubin ≧2.0 × ULN to ≦3.0 × ULN for |
| | >7 consecutive days. |
| | AST or ALT CTCAE Grade ≧3 in conjunction with blood |
| | bilirubin CTCAE Grade ≧2 of any duration |
| | ≧CTCAE grade 3 total bilirubin |
| | CTCAE grade 3 AST or ALT for >7 consecutive days |
| | CTCAE grade 3 GGT elevation in conjunction with CTCAE |
| | Grade ≧3. AST and/or ALT |
| | CTCAE grade 4 AST or ALT |
| | CTCAE grade 4 GGT elevation in conjunction with CTCAE |
| | Grade ≧3. AST and/or ALT |
| Endocrine | ≧Grade 3 hyperglycemia (>13.89 mmol/L or 250 mg/dL |
| (Hyperglycemia is NOT graded | FPG) (confirmed with a repeat FPG within 24 hours) |
| per CTCAE v4.03) | |
| Metabolic/Laboratory | CTCAE grade 3 asymptomatic amylase and/or lipase |
| | >7 consecutive days |
| | CTCAE grade 4 asymptomatic amylase and/or lipase |
| Pancreatitis | ≧CTCAE grade 2 |
| Cardiac | Cardiac toxicity ≧CTCAE grade 3 or cardiac event that is |
| | symptomatic or requires medical intervention |
| | Clinical signs of cardiac disease, such as unstable angina |
| | or myocardial infarction, or Troponin ≧CTCAE grade 3 |
| Mood alteration | CTCAE grade 2 mood alteration that does not resolve to |
| | ≦grade 1 within 14 days despite medical treatment (for |
| | Anxiety only, if worsened from baseline) |
| | ≧CTCAE grade 3 mood alteration |
| | CTCAE Grade 3 rash for >7 consecutive days despite skin |
| | toxicity treatment |
| | CTCAE Grade 4 rash |
| Other adverse events | ≧CTCAE grade 3 adverse events (excluding ≧CTCAE |
| | grade 3 elevations in alkaline phosphatase) |
| | ≧CTCAE grade 3 vomiting/nausea ≧48 hrs, despite the |
| | use of anti-emetic therapy |
| | ≧CTCAE grade 3 diarrhea ≧48 hrs, despite the use of |
| | anti-diarrheal therapy |
| | CTCAE grade 3 fatigue (asthenia) for >7 consecutive days |
| | CTCAE grade 4 fatigue (asthenia) |
| | CTCAE grade 3 stomatitis/mucositis ≧3 days, despite the |
| | use of topical corticosteroids and analgesic mouth |
| | treatments |
| | CTCAE grade 4 stomatitis/mucositis |

Further DLTs include (a) the inability to take 75% or more of the planned daily doses from Days 8-35 in Cycle 1 due to treatment-related adverse events in the COMPOUND A Arm 1 (i.e. >7 days of partial or no dose of COMPOUND A or its monotosylate salt or abiraterone acetate in Days 8-35); or (b) the inability to take 75% or more of the planned daily doses from Days 8-35 in Cycle 1 due to treatment-related AEs in the COMPOUND C Arm 1 (i.e. >7 days of partial or no dose of COMPOUND C or its monohydrochloride salt or abiraterone acetate in Days 8-35).

“MTD” is defined as the highest drug dosage that does not cause medically unacceptable dose-limiting toxicities in more than 35% of the treated patients during the first cycle of treatment. Typically the MTD is a tested dose with a maximum probability of targeted toxicity (DLT rate between 16%-35%). “RDE” is the dose that is recommended for further use in the expansion part of this clinical study. The RDE may be determined to be the same as the MTD. The declared RDE may also be lower than the MTD if the evolving safety profile (long term or overall) along with other assessments such as PK suggest a better safety profile without substantial loss of benefit in exposure/activity.

In the Second Phase (Dose Expansion) of the clinical trial, the primary objective is to assess the anti-tumor activity of the combinations (i.e., abiraterone acetate+COMPOUND A or its monotosylate salt and abiraterone acetate+COMPOUND C or its monohydrochloride salt) in patients diagnosed with castration-resistant prostate cancer patients and after failure of abiraterone acetate monotherapy. A further variable objective is also the proportion of patients in the FAS, with a PSA decrease from baseline of at least 30% at Week 12 or later. Patients are treated with the COMPOUND A Combination (COMPOUND A Arm 2 Group) or the COMPOUND C Combination (COMPOUND C Arm 2 Group) at the MTD or RDE determined in the First Phase. At the time point when the second phase starts, patients from earlier cohorts can be dose-escalated up to the MTD if all of the following criteria are met:
(a) Patient has been treated for least 4 months (4 cycles);
(b) No treatment related toxicity has occurred with the current dose level, resulting in treatment reduction or interruption or discontinuation in the previous 2 cycles.
   These patients repeat the schedule of safety assessments as done for cycle 1 (starting at Day 1) for 1 cycle. Dose escalation is done only after the patient completes the ongoing cycle.

All treatment cycles in the COMPOUND A Arm 2 group consist of 28 calendar days during which COMPOUND A or its monotosylate salt and prednisone are given to each patient twice daily an abiraterone acetate is given once daily. All treatment cycles in the COMPOUND C Arm 2 group consist of 28 calendar days during which COMPOUND C or its monohydrocloride salt and abiraterone acetate are given once daily and prednisone is given twice daily. The last day of a complete treatment cycle is Day 28.

Patients are treated until disease progression, unacceptable toxicity, death or discontinuation from the study treatment due to any other reason. No patients are replaced during the second phase.

Efficacy

In the first and second phase, efficacy may be assessed by decline change in PSA level, time to progression of PSA, radiological progression-free survival, radiological response and/or overall survival.

PSA is measured regularly to calculate PSA decline and time to PSA progression. PSA progression is defined as per PCWG2 as follows:
(a) For patients with decline in PSA levels from baseline: Time from start of therapy to first PSA increase that is ≧25% above the nadir and ≧2 ng/mL above the nadir, and which is confirmed by a second value 3 or more weeks later (ie, a confirmed rising trend) sequentially.
(b) For patients with no decline in PSA levels from baseline: Time from start of therapy to first PSA increase that is ≧25% above baseline and ≧2 ng/mL above baseline after 12 weeks.

Time to PSA progression (TTpP) is calculated and defined according to PCWG2 as follows:
(a) for patients whose PSA has initially decreased, TTpP is defined as the time from start of treatment to the first PSA increase that is ≧25% above the nadir and ≧2 ng/mL above the nadir and which is confirmed by a second value 3 or more weeks later sequentially.
(b) for patients whose PSA has not decreased, TTpP is defined as the time from start of treatment to the first PSA increase that is ≧25% above the nadir and ≧2 ng/mL above the baseline.
   Increase in PSA within the first 12 weeks of treatment will not be considered as progression (unless unequivocal sign of progression).

Radiological progression-free survival is defined per PCWG2 as the time from start of treatment until progression (based on local assessment) or death from any cause. Progression is defined as the occurrence of either tumor progression in soft tissue according to RECIST 1.1 or progression in bone. Progression on Bone scans is defined as the appearance of at least 2 new lesions as compared to the previous scan. For bone scans, progression at the first assessment (week 12) is defined as at least 2 new lesions, is confirmed by a subsequent scan 6 or more weeks later showing at least 2 additional new bone lesions as compared to the first assessment scan as per PCWG2. For subsequent evaluations, according to PCWG2, bone progression is defined as at least 2 new lesions as compared to the previous scan, should be confirmed by a subsequent scan 6 or more weeks later. Progression on CT scan at first assessment are confirmed by a subsequent scan 6 or more weeks later as per PCWG2. Progression on CT scans (soft tissues) and progression on bone scans are analyzed separately.

Radiological response refers to the best overall response according to RECIST 1.1.

Overall survival is defined as the time from start of treatment to the date of death due to any cause.

Efficacy may be preliminarily assessed based upon interim study data as known to one of ordinary skill in the art.

Follow-Up

All patients have safety evaluations for 30 days after last dose of study treatment.

If a patient did not discontinue study treatment due to disease progression, death, start of new anti-neoplastic therapies, lost to follow-up, or withdrawal of consent to efficacy follow-up, then tumor assessments are continuing to be performed every 12 weeks until the start of new anti-cancer therapy, disease progression, death, lost to follow-up, or withdrawal of consent to efficacy follow-up.

All patients are followed for survival status every 3 months regardless of treatment discontinuation reason until death, lost to follow-up, or withdrawal of consent to survival follow-up. Additional survival assessments may be performed if a survival update is required for an interim assessment.

### Example 2

### Clinical Study

A clinical study using (a) a phosphatidylinositol 3-kinase inhibitor that is either COMPOUND A or its monotosylate salt or COMPOUND C or its hydrochloride salt, in combination with (b) abiraterone acetate or a pharmaceutically acceptable salt thereof, and (c) prednisone for treatment of patients with castration-resistant prostate cancer after failure of abiraterone acetate therapy is investigated.

An open-label, unblinded study of the combination comprising (a) either COMPOUND A or its monotosylate salt or COMPOUND C or its hydrochloride salt, (b) abiraterone acetate or a pharmaceutically acceptable salt thereof, and (c) prednisone is conducted in patients who are diagnosed with a castration-resistant prostate cancer and after failure of abiraterone acetate therapy. In the first phase, a dose-escalation study is conducted to determine the maximal tolerated dose (MTD) and/or recommended dose for expansion (RDE) of both (a) COMPOUND A or its monotosylate salt in combination with abiraterone acetate and prednisone (hereinafter referred to as “COMPOUND A Arm 1”), and (b) COMPOUND C or its monohydrochloride salt in combination with abiraterone acetate and prednisone (hereinafter referred to as “COMPOUND C Arm 1”) in patients diagnosed with castration-resistant prostate cancer after failure of abiraterone acetate therapy. Approximately 15 to 25 patients are enrolled in the COMPOUND A Arm 1, and approximately 10 to 20 patients are enrolled in the COMPOUND C Arm 1 in the first phase. In the second phase, a dose expansion phase is conducted to investigate the antitumor activity of both (a) COMPOUND A or its monotosylate salt in combination with abiraterone acetate and prednisone (hereinafter referred to as “COMPOUND A Arm 2”), and (b) COMPOUND C or its monohydrochloride salt in combination with abiraterone acetate and prednisone (hereinafter referred to as “COMPOUND C Arm 2”) in patients diagnosed with castration-resistant prostate cancer after failure of treatment with abiraterone acetate therapy. Approximately 35 to 45 patients are enrolled in the COMPOUND A Arm 2, and approximately 35 to 45 patients are enrolled in the COMPOUND C Arm 2 in the second phase. Patients are treated until disease progression, unacceptable toxicity, death, or discontinuation from the study treatment due to any other reason.

For the clinical study, “failure of abiraterone acetate therapy” is defined as progression of the castration-resistant prostate cancer after treatment with abiraterone acetate therapy alone or abiraterone acetate therapy in combination with prednisone or corticosteroids administered by topical application, inhalation, eye drops, or local injection. In the first phase, patients have a documented progressive disease to prior abiraterone acetate therapy according to Prostate Cancer Working Group 2 (PCWG2) criteria according to at least one of the following criteria: (a) patient who progressed based solely upon rising levels of the serum marker Prostate-Specific Antigen (PSA), wherein the sequence of rising values occurs on three (3) consecutive occasions of at least 1 week intervals and having 5.0 ng/mL minimum level for entry into the study, (b) patient who manifest disease progression per Response Evaluation Criteria in Solid Tumors (“RECIST”) independent of PSA level, and/or (c) patient with bone only progression according to PCWG2. In the second phase, patients must have had a prior Prostate-Specific Antigen response followed by documented Prostate-Specific Antigen (“PSA”) progression on abiraterone acetate monotherapy. “PSA progression” is defined as a sequence of rising levels of PSA on three (3) consecutive occasions of at least 1 week intervals and having 5.0 ng/mL minimum level for entry into the study,

Potential patients are screened within 7 to 28 day days prior to treatment start. The following inclusion and exclusion criteria define those patients eligible for the study:

Inclusion Criteria:

Patients eligible for this study are those satisfying all of the following criteria:
Adult males 18 years old
Eastern Cooperative Oncology Group Performance Status ≦2
Patient having a castrate level of testosterone (<=50 ng/dL or 1.7 nmol/L). Castrate status must be maintained by continued Gonadotropin-releasing hormone (GnRH) analogues unless patient has undergone surgical orchiectomy).
Histologically or cytologically confirmed diagnosis of advanced or metastatic prostate cancer.
Advanced or metastatic castration-resistant prostate cancer progression after abiraterone acetate failure
Patients having no more than 2 lines of prior chemotherapies including cytotoxic agents
Discontinuation of all anti-androgen, anti-neoplastic or investigational treatment >=4 weeks (6 weeks for bicalutamide). Discontinuation of abiraterone acetate is not required.
Patient has adequate bone marrow and organ function as defined by the following laboratory values:
   Absolute Neutrophil Count (ANC) ≧1.0×10⁹/L
   Platelets ≧100×10⁹/L (in case of transfusion stable for ≧14 days prior to treatment start)
   Hemoglobin ≧9.0 g/kL (in case of transfusion stable for ≧14 days prior to treatment start)
   INR ≦2
   Serum Creatinine ≦1.5×ULN and creatinine clearance >45 mL/min
   Potassium, calcium (corrected for serum albumin) and magnesium within normal limits (WNL).
   Alanine aminotransferase (ALT) and aspartate aminotransferase (AST) equal or below the upper limit of normal range (or <3.0×ULN in case of liver metastases).
   Total serum Bilirubin equal or below the upper limit of normal range (or ≦1.5×ULN if liver metastases are present; or total bilirubin ≦3.0×ULN with direct bilirubin within normal range in patiens with well documented Gilbert Syndrome which is defined as presence of several episodes of unconjugated hyperbilirubinemia with normal results from CBC count (including normal reticulocyte count and blood smear), normal liver function test results, and absence of other contributing disease processes at the time of diagnosis.
   Fasting plasma glucose (FPG)≦120 mg/dL or ≦6.7 mmol/L
   HbA1c<=8%.
Recovery from all treatment-related toxicity to Grade ≦1 within 4 weeks prior to treatment start with exception of alopecia, organ functions and bone marrow as described above.
Agree to use effective contraception during the study and for at least 16 weeks after discontinuation.
Patient is able to swallow and retain oral medication

Additional inclusion criteria for First Phase (dose escalation part) only):
Patient having documented progressive disease to the previous line of therapy according to PCWG2 criteria:
   Patient who progressed based solely on Prostate-Specific Antigen (PSA) rising levels, should have had a sequence of rising values on 3 consecutive occasions of at least 1 week intervals and should have 5.0 ng/mL minimum level for entry.
   Patients who manifested disease progression per RECIST are eligible independent of PSA.
   Patients with bone only progression according to PCWG2.

Additional inclusion criteria for Second Phase (dose expansion part) only:
Patient having a prior Prostate-Specific Antigen (PSA) response, followed by documented PSA progression on abiraterone acetate therapy.

“PSA progression” is defined as a sequence of rising values on 3 consecutive occasions of at least 1 week intervals and should have 5.0 ng/mL minimum level for entry.
Abiraterone Acetate is the last treatment prior to enrolment in the dose expansion part of the study and no other anticancer therapy is allowed between Abiraterone Acetate and study entry.

Exclusion Criteria:

Patients eligible for this study are those that do not meet any of the following criteria:
Previous treatment with PI3K pathway inhibitors (e.g. PI3K, AKT, mTOR inhibitor), ketoconazole, CYP17 inhibitors (exception of AA), or MDV3100.
Known hypersensitivity and/or contraindication to any of the study medications or their excipients.
Patient has a concurrent malignancy or malignancy within 3 years of study enrollment (with exception of adequately treated basal or squamous cell carcinoma, non-melanomatous skin cancer or curatively resected cervical cancer).
Patient having active uncontrolled or symptomatic Central Nervous System

(CNS) metastases. A patient with controlled and asymptomatic CNS metastases may participate in this trial. As such, the patient must have completed any prior treatment for CNS metastases >28 days (including radiotherapy and/or surgery) prior to start of treatment in this study and should not be receiving chronic corticosteroid therapy for the CNS metastases.
Patient has received wide field radiotherapy (including therapeutic radioisotopes such as strontium 89) ≦28 days or limited field radiation for palliation ≦14 days prior to starting study drug or has not recovered from side effects of such therapy.
Patient has had major surgery within 14 days prior to starting study drug or has not recovered from major side effects.
Patient has not recovered to grade 1 or better (except alopecia, bone marrow and organ functions listed above) from related side effects of any prior antineoplastic therapy.
Inadequately controlled hypertension (e.g. systolic blood pressure >=160 mmHg or diastolic blood pressure >=95 mmHg)
Patient has active cardiac disease or a history of cardiac dysfunction including any of the following:
   Severe or unstable angina pectoris within 6 months prior to study entry
   Symptomatic pericarditis
   Documented myocardial infarction or arterial thrombotic events within 6 months prior to study entry
   History of documented congestive heart failure (New York Heart Association functional classification III-IV)
   Documented cardiomyopathy
Patient has a Left Ventricular Ejection Fraction (LVEF)<50% as determined by Multiple Gated acquisition (MUGA) scan or echocardiogram (ECHO)
Patient has any of the following cardiac conduction abnormalities
   Ventricular arrhythmias except for benign premature ventricular contractions
   Supraventricular and nodal arrhythmias requiring a pacemaker or not controlled with medication
   Conduction abnormality requiring a pacemaker
   Other cardiac arrhythmia not controlled with medication
Patient has a QTcF >480 msec on the screening ECG (using the QTcF formula), has a short/long QT syndrome, or history of QT prolongation/Torsades de Pointes
Patient having a QT interval corrected for heart rate using Fridericia's formula (QTcF) >480 msec on the screening Electrocardiogram (using the QT interval corrected for heart rate using Fridericia's formula), has a short/long QT syndrome, or history of QT prolongation/Torsades de Pointe
Patient is currently receiving treatment with medication that has a known risk to prolong the QT interval or inducing Torsades de Pointes, and the treatment cannot be discontinued or switched to a different medication prior to treatment start.
Patient is currently receiving increasing or chronic treatment (>5 days) with corticosteroids or another immunosuppressive agent, as chronic administration of corticosteroids (>5 days) can induce CYP3A4. The following corticosteroids are permitted:
c. Prednisone 5 mg BID as part of the study treatment;
d. Topical applications (e.g., rash), inhaled sprays (e.g., obstructive airways diseases), eye drops or local injections (e.g., intra-articular)
Patient is currently receiving warfarin or other coumarin derived anti-coagulant, for treatment, prophylaxis or otherwise. Therapy with heparin, low molecular weight heparin (LMWH), or fondaparinux is allowed
Patient is currently receiving treatment with drugs known to be moderate or strong inhibitors or inducers of isoenzyme CYP3A. The patient must have discontinued strong inducers for at least one week and must have discontinued strong inhibitors before the treatment is initiated.
Patient having impairment of gastrointestinal (GI) function or GI disease that may significantly alter the absorption of study drugs (e.g., ulcerative diseases, uncontrolled nausea, vomiting, diarrhea, malabsorption syndrome, or small bowel resection)
Patient has any other concurrent severe and/or uncontrolled medical condition that would, in the investigator's judgment contraindicate patient participation in the clinical study (e.g. active or uncontrolled severe infection, chronic pancreatitis, active or symptomatic hepatitis, chronic obstructive or restrictive pulmonary disease including dyspnea at rest, interstitial lung disease, uncontrolled high blood pressure, adrenal insufficiency, etc.)
Patient has a history of non-compliance to medical regimen or inability to grant consent
Patient has a known history of HIV infection (testing not mandatory) infection
Patient is concurrently using other approved or investigational antineoplastic agent.
Patient selects a response of “1, 2 or 3” to question number 9 on the PHQ-9 questionnaire regarding potential for suicidal thoughts or ideation (independent of the total score of the PHQ-9)
Patient having a medically documented history of or active major depressive episode, bipolar disorder (I or II), obsessive-compulsive disorder, schizophrenia, a history of suicidal attempt or ideation, or homicidal ideation (e.g. risk of doing harm to self or others) and/or (a) patient has ≧CTCAE grade 3 anxiety, (b) Patient has a Generalized Anxiety Disorder 7-item scale (GAD-7) mood scale score ≧15, or (c) Patient has a score ≧12 on the Patient Health Questionaire-9 item (PHQ-9) questionnaire.
Patient is consuming Seville oranges, grapefruit, grapefruit hybrids, pummelos and exotic citrus fruits (as well as their juices) during the last 7 days prior to start of treatment. Regular orange juice is permitted.
Sexually active males not willing to use a condom during the whole course of the study and for 16 weeks after stopping treatment.
Patients who has experienced dose reductions and/or treatment interruptions due to abiraterone acetate related toxicities (i.e. serious adverse events, adverse events, liver toxicities during abiraterone acetate treatment

Additional exclusion criteria for Second Phase (dose expansion part) only:
Patients who has received any anticancer systemic therapy between abiraterone acetate and study entry
   During the screening, the patient's diagnosis and extent of cancer (including staging, histology/cytology and sites of disease at study entry, PSA history and PSA level), demography, medical history, prior antineoplastic therapies, all medications and significant non-drug therapies taken within 28 days before first dose, and other physical characteristics (e.g, vital signs, height, weight, physical examination, ECOG, laboratory evaluations of blood, ECG, cardiac imaging, CT/MRI and bone scan assessments of tumors and additional radiological assessments if clinically indicated, and mood) are assessed.

In the First Phase (Dose Escalation) of the clinical trial, the primary objective is to determine (a) the MTD and/or RDE of COMPOUND A or its monotosylate salt in combination with abiraterone acetate and (b) the MTD and/or RDE of COMPOUND C or its monohydrochloride salt in combination with abiraterone acetate. A secondary objective is to determine the percent change from baseline in PSA at Week 12 as well as the best percentage change from baseline at any time as calculated using water fall plots. The proportion of patients with PSA decrease from baseline of at least 30% at Week 12 or later is calculated for patients of the first phase.

In the COMPOUND A Arm 1 group, patients are provisionally administered each of the following: (a) 200 mg of COMPOUND A, per oral, twice daily (B.I.D.) for a total daily dose of 400 mg; (b) 1000 mg abiraterone acetate, per oral, once daily (Q.D.); and (c) 5 mg prednisone, per oral, twice daily (B.I.D.) for a total daily dose of 10 mg. In the COMPOUND C Arm 1 group, patients are provisionally administered each of the following: (a) 60 mg of COMPOUND C, per oral, once daily (Q.D.), (b) 1000 mg abiraterone acetate, per oral, once daily (Q.D.); and (c) 5 mg prednisone, per oral, twice daily (B.I.D.) for a total daily dose of 10 mg. COMPOUND A may be administered in the form of its free base or its monotosylate salt form. A complete treatment cycle is defined as 35 calendar days consisting of (a) a 7-day run-in period during which abiraterone acetate or its pharmaceutically acceptable salt is given once daily and prednisone is given twice daily and (b) 28 days combination treatment period during which COMPOUND A or its monotosylate salt and prednisone are given twice daily and abiraterone acetate or its pharmaceutically acceptable salt is given once daily. Combination treatment with COMPOUND A or its monotosylate salt begins on Day 8 of Cycle 1. The last day of the first treatment cycle is Day 35. All subsequent treatment cycles for COMPOUND A Arm 1 consist of 28 calendar days during which COMPOUND A or its monotosylate salt and prednisone are given twice daily and abiraterone acetate is given once daily.

In the COMPOUND C Arm 1 group, patients are provisionally administered each of the following: (a) 60 mg of COMPOUND C, per oral, once daily (Q.D.); (b) 1000 mg abiraterone acetate, per oral, once daily (Q.D.); and (c) 5 mg prednisone, per oral, twice daily (B.I.D.) for a total daily dose of 10 mg. COMPOUND C may be administered in the form of its free base or its monohydrochloride salt form. A complete treatment cycle consists of 28 calendar days during which COMPOUND C or its monohydrochloride salt and abiraterone acetate are given once daily and prednisone is given twice daily. The last day of the complete treatment cycle is Day 28.

A cohort of approximately 3-6 patients is treated at the next dose level. Each cohort is consisting of newly enrolled patients. To account for the potential of drop-outs during the first cycle of treatment (e.g., due to early disease progression), up to up to 2 additional patients may be enrolled, if these additional patients can be treated within 14 days after the third patient is first dosed with the COMPOUND A or its monotosylate salt. The dose levels for the dose-escalation phase of the clinical study are defined as follows for the COMPOUND A Arm 1 Group:

| Dose | | Abiraterone | |
|---|---|---|---|
| level | COMPOUND A | acetate | Prednisone |
| Dose | 200 mg BID | 1000 mg (QD) | 5 mg BID (2 × 5 mg for a |
| Level 1 | (2 × 200 mg | | total of 10 mg a day) |
| | for total of 400 | | |
| | mg a day) | | |
| Dose | 300 mg BID | 1000 mg (QD) | 5 mg BID (2 × 5 mg for a |
| Level 2 | (2 × 300 mg | | total of 10 mg a day) |
| | for total of 600 | | |
| | mg a day) | | |
| Dose | 400 mg BID | 1000 mg (QD) | 5 mg BID (2 × 5 mg for a |
| Level 3 | (2 × 400 mg | | total of 10 mg a day) |
| | for total of 800 | | |
| | mg a day) | | |

Further, the dose levels for the dose-escalation phase of the clinical study are defined as follows for the COMPOUND C Arm 1 Group:

| Dose | | Abiraterone | |
|---|---|---|---|
| level | COMPOUND C | acetate | Prednisone |
| Dose | 60 mg (QD) | 1000 mg (QD) | 5 mg BID (2 × 5 mg for a |
| Level 1 | | | total of 10 mg a day) |
| Dose | 80 mg (QD) | 1000 mg (QD) | 5 mg BID (2 × 5 mg for a |
| Level 2 | | | total of 10 mg a day) |
| Dose | 100 mg (QD) | 1000 mg (QD) | 5 mg BID (2 × 5 mg for a |
| Level 3 | | | total of 10 mg a day) |

In the first phase, the individual dose is dependent on the current dose level at which the patient enters the study. A five-parameter adaptive Bayesian logistic regression model guided by escalation with overdose control (EWOC) principle is used for dose level selection and for determination of the MTD and/or RDE of the COMPOUND A combination treatment and the COMPOUND C combination treatment. Patients are considered evaluable for the dose-determining set if the patient experiences a dose-limiting toxicity (DLT) during cycle 1, or meets the minimum treatment exposure and safety evaluations required as defined by the following criteria:
(a) Patients in the COMPOUND A Arm 1 group: The minimum exposure criterion is at least 21 of the 28 full daily planned doses of abiraterone acetate and COMPOUND A or its monotosylate salt between Day 8 and Day 35 of Cycle 1.
(b) Patients in the COMPOUND C Arm 1 group: The minimum exposure crtierion is at least 21 of the 28 full daily planned doses of abiraterone acetate and COMPOUND C or its monohydrocloride salt between Day 1 and Day 28 of Cycle 1.
(c) Patients should have completed the required safety evaluations during Cycle 1 to an extent that the investigator or physician can determine that no unacceptable toxicities occurred in Cycle 1 for this dosing cohort.

To determine the dose level for the next cohort in the dose-escalation, the available toxicity information (including dose limiting toxicities and adverse events that are not dose limiting toxicities), pharmacokinetics, pharmacodynamics, efficacy information and recommendations from the five-parameter adaptive Bayesian logistic regression model (BLRM) are evaluated. A 5 parameter BLRM for each combination treatment is fitted on Cycle 1 dose-limiting toxciity data (i.e., absence or presence of DLT) accumulated throughout the dose-escalation to model the dose-toxicity relationship of abiraterone acetate and either COMPOUND A or COMPOUND C when given in combination. The dose for the next cohort is not exceeding the maximum dose allowed by the five-parameter adaptive Bayesian logistic regression model. After completion of cycle 1 of each cohort, the BLRM is used to make recommendations about the next dose level, with the following exceptions:
If 2 of the first 3 patients in a cohort experience a dose limiting toxicity (DLT), further enrollment to that cohort is suspended. The BLRM is updated with this new information before additional patients are enrolled to the study.
If a decision is made to escalate to a higher dose level, but one or more additional patient(s) are treated at the preceding dose level experiences a dose limiting toxicity (DLT), in cycle 1, then the BRLM is updated before any additional patient is enrolled to the higher dose level.
   Dose escalation is continued until identification of the MTD and/or RDE.

A dose-limiting toxicity (DLT) is defined as an adverse event or abnormal laboratory value assessed as unrelated to disease, disease progression, inter-current illness, or concomitant medications that occurs within the first cycle and meets any of the criteria included below. National Cancer Institute Common Terminology Criteria for Adverse events (NCI CTCAE) version 4.03, which is hereby incorporated by reference in its entirety, will be used for all grading. For the purpose of dose-escalation decisions, the following DLTs are considered for these drug combinations:

TABLE 2

| TOXICITY | ANY OF THE FOLLOWING CRITERIA |
|---|---|
| Hematologic | CTCAE grade 4 neutropenia |
| | CTCAE grade 4 thrombocytopenia |
| | Febrile neutropenia (ANC <1.0 × 10⁹/L, fever ≧38.3° C.) |
| | CTCAE Grade ≧3. |
| Renal | ≧CTCAE grade 3 serum creatinine |
| Hepatic | Total bilirubin ≧2.0 × ULN to ≦3.0 × ULN for |
| | >7 consecutive days. |
| | AST or ALT CTCAE Grade ≧3 in conjunction with blood |
| | bilirubin CTCAE Grade ≧2 of any duration |
| | ≧CTCAE grade 3 total bilirubin |
| | CTCAE grade 3 AST or ALT for >7 consecutive days |
| | CTCAE grade 3 GGT elevation in conjunction with CTCAE |
| | Grade ≧3. AST and/or ALT |
| | CTCAE grade 4 AST or ALT |
| | CTCAE grade 4 GGT elevation in conjunction with CTCAE |
| | Grade ≧3. AST and/or ALT |
| Endocrine | ≧Grade 3 hyperglycemia (>13.89 mmol/L or 250 mg/dL |
| (Hyperglycemia is NOT graded | FPG) (confirmed with a repeat FPG within 24 hours) |
| per CTCAE v4.03) | |
| Metabolic/Laboratory | CTCAE grade 3 asymptomatic amylase and/or lipase |
| | >7 consecutive days |
| | CTCAE grade 4 asymptomatic amylase and/or lipase |
| Pancreatitis | ≧CTCAE grade 2 |
| Cardiac | Cardiac toxicity ≧CTCAE grade 3 or cardiac event that is |
| | symptomatic or requires medical intervention |
| | Clinical signs of cardiac disease, such as unstable angina |
| | or myocardial infarction, or Troponin ≧CTCAE grade 3 |
| Mood alteration | CTCAE grade 2 mood alteration that does not resolve to |
| | ≦grade 1 within 14 days despite medical treatment (for |
| | Anxiety only, if worsened from baseline) |
| | ≧CTCAE grade 3 mood alteration |
| Rash | CTCAE Grade 3 rash for >7 consecutive days despite skin |
| | toxicity treatment |
| | CTCAE Grade 4 rash |
| Other adverse events | ≧CTCAE grade 3 adverse events (excluding ≧CTCAE |
| | grade 3 elevations in alkaline phosphatase) |
| | ≧CTCAE grade 3 vomiting/nausea ≧48 hrs, despite the |
| | use of anti-emetic therapy |
| | ≧CTCAE grade 3 diarrhea ≧48 hrs, despite the use of |
| | anti-diarrheal therapy |
| | CTCAE grade 3 fatigue (asthenia) for >7 consecutive days |
| | CTCAE grade 4 fatigue (asthenia) |
| | CTCAE grade 3 stomatitis/mucositis ≧3 days, despite the |
| | use of topical corticosteroids and analgesic mouth |
| | treatments |
| | CTCAE grade 4 stomatitis/mucositis |

Further DLTs include (a) the inability to take 75% or more of the planned daily doses from Days 8-35 in Cycle 1 due to treatment-related adverse events in the COMPOUND A Arm 1 (i.e. >7 days of partial or no dose of COMPOUND A or its monotosylate salt or abiraterone acetate in Days 8-35); or (b) the inability to take 75% or more of the planned daily doses from Days 8-35 in Cycle 1 due to treatment-related AEs in the COMPOUND C Arm 1 (i.e. >7 days of partial or no dose of COMPOUND C or its monohydrochloride salt or abiraterone acetate in Days 8-35).

For patients who do not tolerate the defined dosing schedule, dose adjustments are permitted once a DLT is determined. For subjects experiencing a DLT or intolerable toxicity, the treatment causing the DLT should be withheld until toxicity resolves to Grade 1 or better. A maximum of 2 dose reductions for each drug are allowed and once reduced, it cannot be reescalated. If dose interruptions last for greater than 28 consecutive days, the patient is discontinued from the study. Dose modification for COMPOUND A or its monotosylate salt is permitted as follows:

| Dose levels | Dose and schedule | | |
|---|---|---|---|
| Current dose | 200 mg BID | 300 mg BID | 400 mg BID |
| Decrease 1 dose | 100 mg BID | 200 mg BID | 300 mg BID |
| level | | | |
| Decrease 2 dose | Discontinue | 100 mg BID | 200 mg BID |
| levels | | | |
| Decrease 3 dose | Discontinue | Discontinue | 100 mg BID |
| levels | | | |

“MTD” is defined as the highest drug dosage that does not cause medically unacceptable dose-limiting toxicities in more than 35% of the treated patients during the first cycle of treatment. Typically the MTD is a tested dose with a maximum probability of targeted toxicity (DLT rate between 16%-35%). “RDE” is the dose that is recommended for further use in the expansion part of this clinical study. The RDE may be determined to be the same as the MTD. The declared RDE may also be lower than the MTD if the evolving safety profile (long term or overall) along with other assessments such as PK suggest a better safety profile without substantial loss of benefit in exposure/activity.

In the Second Phase (Dose Expansion) of the clinical trial, the primary objective is to assess the anti-tumor activity of the combinations (i.e., abiraterone acetate+COMPOUND A or its monotosylate salt and abiraterone acetate+COMPOUND C or its monohydrochloride salt) in patients diagnosed with castration-resistant prostate cancer patients and after failure of abiraterone acetate monotherapy. A further variable objective is also the proportion of patients in the FAS, with a PSA decrease from baseline of at least 30% at Week 12 or later. Patients are treated with the COMPOUND A Combination (COMPOUND A Arm 2 Group) or the COMPOUND C Combination (COMPOUND C Arm 2 Group) at the MTD or RDE determined in the First Phase. At the time point when the second phase starts, patients from earlier cohorts can be dose-escalated up to the MTD if all of the following criteria are met:
(a) Patient has been treated for least 4 months (4 cycles);
(b) No treatment related toxicity has occurred with the current dose level, resulting in treatment reduction or interruption or discontinuation in the previous 2 cycles.
   These patients repeat the schedule of safety assessments as done for cycle 1 (starting at Day 1) for 1 cycle. Dose escalation is done only after the patient completes the ongoing cycle.

All treatment cycles in the COMPOUND A Arm 2 group consist of 28 calendar days during which COMPOUND A or its monotosylate salt and prednisone are given to each patient twice daily an abiraterone acetate is given once daily. All treatment cycles in the COMPOUND C Arm 2 group consist of 28 calendar days during which COMPOUND C or its monohydrocloride salt and abiraterone acetate are given once daily and prednisone is given twice daily. The last day of a complete treatment cycle is Day 28.

Patients are treated until disease progression, unacceptable toxicity, death or discontinuation from the study treatment due to any other reason. No patients are replaced during the second phase.

Efficacy

In the first and second phase, efficacy may be assessed by decline change in PSA level, radiological progression-free survival, radiological response and/or overall survival.

PSA is measured regularly to calculate PSA decline and time to PSA progression. PSA progression is defined as per PCWG2 as follows:
(a) For patients with decline in PSA levels from baseline: Time from start of therapy to first PSA increase that is ≧25% above the nadir and ≧2 ng/mL above the nadir, and which is confirmed by a second value 3 or more weeks later (ie, a confirmed rising trend) sequentially.
(b) For patients with no decline in PSA levels from baseline: Time from start of therapy to first PSA increase that is ≧25% above baseline and ≧2 ng/mL above baseline after 12 weeks.

Radiological progression-free survival is calculated for patients in the dose expansion and is defined per PCWG2 as the time from start of treatment until progression (based on local assessment) or death from any cause. Progression is defined as the occurrence of either tumor progression in soft tissue according to RECIST 1.1 or progression in bone. Progression on Bone scans is defined as the appearance of at least 2 new lesions as compared to the previous scan. Progression in bone as progression in solft tissues are also calculated separately for patients in dose escalation and patients in dose expansion. For bone scans, progression at the first assessment (week 12) is defined as at least 2 new lesions, is confirmed by a subsequent scan 6 or more weeks later showing at least 2 additional new bone lesions as compared to the first assessment scan as per PCWG2. For subsequent evaluations, according to PCWG2, bone progression is defined as at least 2 new lesions as compared to the previous scan, should be confirmed by a subsequent scan 6 or more weeks later. Progression on CT scan at first assessment are confirmed by a subsequent scan 6 or more weeks later as per PCWG2. Progression on CT scans (soft tissues) and progression on bone scans are analyzed separately.

Radiological response refers to the best overall response according to RECIST 1.1.

Overall survival is defined as the time from start of treatment to the date of death due to any cause.

Efficacy may be preliminarily assessed based upon interim study data as known to one of ordinary skill in the art.

Follow-Up

All patients have safety evaluations for 30 days after last dose of study treatment.

If a patient did not discontinue study treatment due to disease progression, death, start of new anti-neoplastic therapies, lost to follow-up, or withdrawal of consent to efficacy follow-up, then tumor assessments are continuing to be performed every 12 weeks until the start of new anti-cancer therapy, disease progression, death, lost to follow-up, or withdrawal of consent to efficacy follow-up.

All patients are followed for survival status every 3 months regardless of treatment discontinuation reason until death, lost to follow-up, or withdrawal of consent to survival follow-up. Additional survival assessments may be performed if a survival update is required for an interim assessment.

### Example 3

### Pre-Clinical Studies of the Combination of 1-(2-Chloro-pyridin-4-yl)-3-(4-methyl-pyridin-3-yl)-imidazolidin-2-one (Compound D) and COMPOUND C or its Hydrochloride Salt

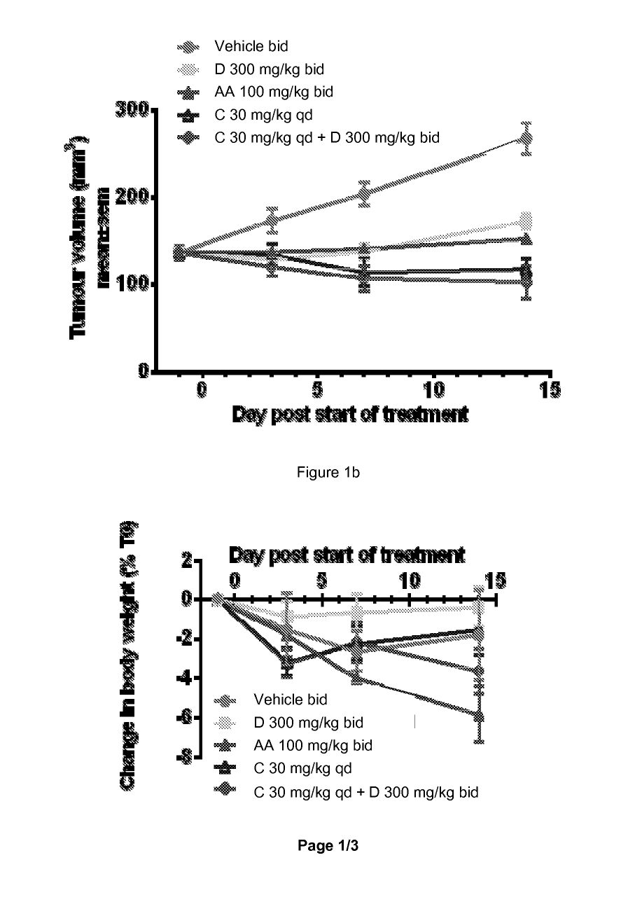

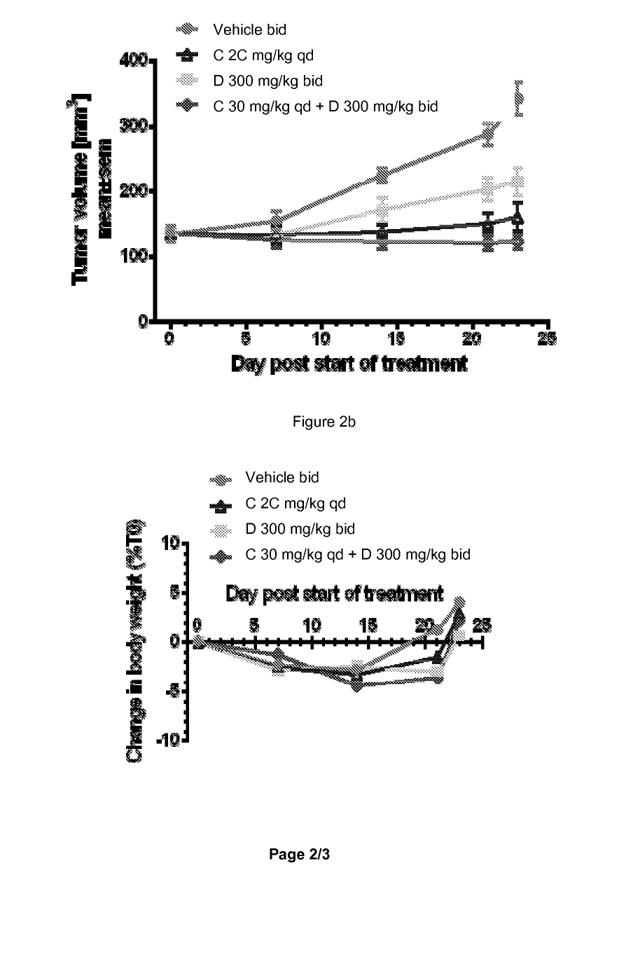

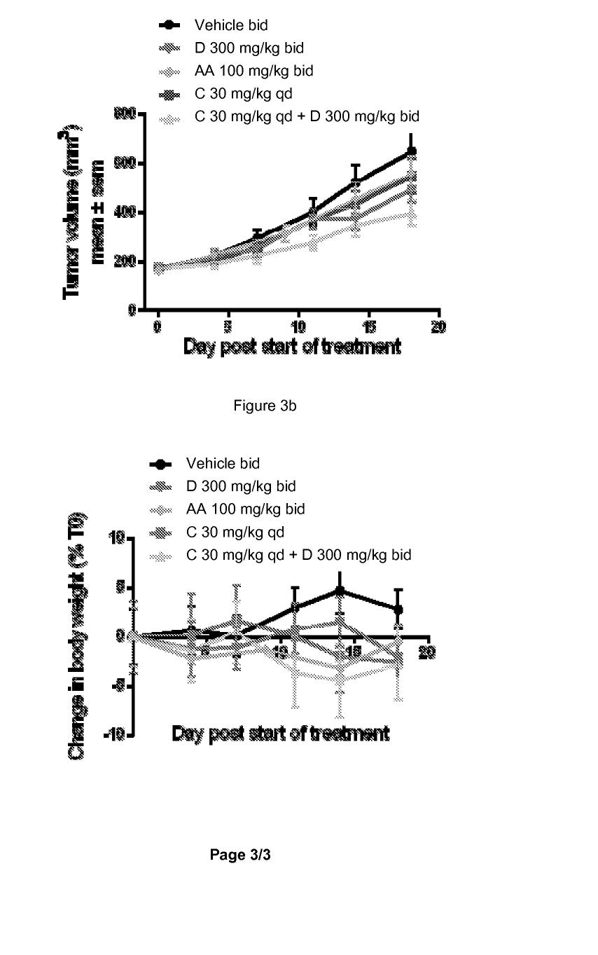

Recently, in vivo anti-tumor efficacy of 1-(2-Chloro-pyridin-4-yl)-3-(4-methyl-pyridin-3-yl)-imidazolidin-2-one was evaluated alone and in combination with COMPOUND C or its hydrochloride salt (a pan PI3K inhibitor) in two human prostate cancer xenograft models, VCap and LnCap, established in SCID mice. The VCap tumors which, express PTEN and over-express wild type AR, were established in chemically-castrated mice. The LnCap tumors which, express mutant AR (T877A) and are PTEN null, were initially established in non-castrated animals which were chemically-castrated at the time of treatment. In two separate experiments with the VCap model, 1-(2-Chloro-pyridin-4-yl)-3-(4-methyl-pyridin-3-yl)-imidazolidin-2-one treatment (300 mg/kg bid) resulted in either slight tumor regression or tumor stasis initially, followed by tumor growth delay, as shown in FIGS. 1-2 and Tables 3-6. The response was comparable to that observed with AA dosed at 100 mg/kg bid. COMPOUND C or its hydrochloride salt dosed at 20 mg/kg qd caused tumor growth delay and at 30 mg/kg qd caused tumor regression. Combination treatment with 1-(2-Chloro-pyridin-4-yl)-3-(4-methyl-pyridin-3-yl)-imidazolidin-2-one (300 mg/kg bid) and COMPOUND C or its hydrochloride salt (either at 20 or 30 mg/kg qd) resulted in slightly greater anti-tumor response than either drug alone. In the LnCap xenograft model single agent treatment with 1-(2-Chloro-pyridin-4-yl)-3-(4-methyl-pyridin-3-yl)-imidazolidin-2-one (300 mg/kg bid), AA (100 mg/kg bid) and COMPOUND C or its hydrochloride salt (30 mg/kg qd) caused marginal tumor growth delay. Combination treatment with 1-(2-Chloro-pyridin-4-yl)-3-(4-methyl-pyridin-3-yl)-imidazolidin-2-one (300 mg/kg bid) plus COMPOUND C or its hydrochloride salt (30 mg/kg qd) resulted in greater anti-tumor response than single agent treatments, as shown in FIG. 3 and Table 7. Thus in all three studies, although the combination did not significantly increase efficacy over single agent response, there was a trend for improvement. And based on the non-statistical method of Clarke (1997) the combination treatment response was additive to synergistic. In addition, all the treatment schedules were well tolerated as measured by body weight changes. These data indicate that combination treatment with 1-(2-Chloro-pyridin-4-yl)-3-(4-methyl-pyridin-3-yl)-imidazolidin-2-one (COMPOUND D) and COMPOUND C or its hydrochloride salt improve response in castrate resistant prostate cancers.

TABLE 3
MIS-32S-12 Day 14 Values

| | | Tumor response | | Host response | | |
|---|---|---|---|---|---|---|
| | | | ΔTumor | | Δbody | |
| | Dose, schedule, | | volume | Δbody | weight | Survival |
| Compound | route | T/C (%) | (mm³) | weight (g) | (%) | (alive/total) |
| Vehicle | 10 ml/kg, bid | 100 | 130.8 ± 17.6 | −0.6 ± 0.6 | −1.8 ± 2.3 | 7/7 |
| | (q12h), p.o. | | | | | |
| D | 300 mg/kg, bid | 27.2*^{$} | 35.6 ± 10.5 | −0.1 ± 0.3 | −0.4 ± 1.0 | 7/7 |
| | (q12h), p.o. | | | | | |
| AA | 100 mg/kg, bid | 11.6* | 15.2 ± 15.9 | −1.6 ± 0.4 | −5.8 ± 1.4 | 7/7 |
| | (q12h), p.o. | | | | | |
| C | 30 mg/kg, qd | Regression | −18.9 ± 6.3 | −0.5 ± 0.3 | −1.5 ± 1.3 | 7/7 |
| | (q24h), p.o. | 13.8%* | | | | |
| C + D | 30 mg/kg, qd + 300 | Regression | −33.5 ± 12.4 | −1.0 ± 0.3 | −3.6 ± 1.2 | 7/7 |
| | mg/kg, bid, p.o. | 24.6%*^{$} | | | | |

Tumors were established in male SCID mice by subcutaneous inoculation of human prostate VCap cells (5 × 10⁶ cells/100 μl HBSS + matrigel 1:1 mixture). On the day cell inoculation animals were chemically castrated by intramuscular injection of 15 μl of Lucrin (3.73 mg/ml). Forty eight days after cell inoculation, treatment was started on day 0 with compounds D, (Abiraterone Acetate, AA), C, and combination of C + D at the doses and schedules listed above (mg/kg). The compounds were dosed orally and compared with control vehicle (0.45% methyl cellulose, 0.1% Tween 80 in water) administered at 10 ml/kg; bid; p.o. Tumor volumes were estimated using the largest diameter (L) and a diameter perpendicular to this (W) according to (L × W²) * π/6. Data are presented as means ± SEM. Statistical analysis was performed using one-way ANOVA with either post hoc Dunnet's test for comparison of treatment versus vehicle control group (*P < 0.05) or the post-hoc Tukey test for intra group analysis (^{$}P < 0.05).

TABLE 4
MIS-32S-12 Day 35 Values

| | | Tumor response | | Host response | | |
|---|---|---|---|---|---|---|
| | | | ΔTumor | | Δbody | |
| | Dose, schedule, | | volume | Δbody | weight | Survival |
| Compound | route | T/C (%) | (mm³) | weight (g) | (%) | (alive/total) |
| Vehicle | 10 ml/kg, bid | 100 | 466.4 ± 27.3 | 0.8 ± 0.5 | 3.1 ± 1.9 | 7/7 |
| | (q12h), p.o. | | | | | |
| D | 300 mg/kg, bid | 65.2*^{;$;#} | 303.9 ± 57.3 | 0.1 ± 0.2 | 0.1 ± 0.8 | 7/7 |
| | (q12h), p.o. | | | | | |
| AA | 100 mg/kg, bid | 34.2*^{;#} | 159.7 ± 57.7 | −1.9 ± 0.6 | −6.6 ± 2.1 | 7/7 |
| | (q12h), p.o. | | | | | |
| C | 30 mg/kg, qd | 5.0*^{;$} | 23.3 ± 21.6 | 0.5 ± 0.6 | 2.0 ± 2.3 | 7/7 |
| | (q24h), p.o. | | | | | |
| C + D | 30 mg/kg, qd + 300 | 5.2*^{;#} | 24.3 ± 11.4 | −1.7 ± 0.5 | −6.1 ± 1.6 | 7/7 |
| | mg/kg, bid, p.o. | | | | | |

Tumors were established in male SCID mice by subcutaneous inoculation of human prostate VCap cells (5 × 10⁶ cells/100 μl HBSS + matrigel 1:1 mixture). On the day cell inoculation animals were chemically castrated by intramuscular injection of 15 μl of Lucrin (3.73 mg/ml). Forty eight days after cell inoculation, treatment was started on day 0 with compounds D, (Abiraterone Acetate, AA), C, and combination of C + D at the doses and schedules listed above (mg/kg). The compounds were dosed orally and compared with control vehicle (0.45% methyl cellulose, 0.1% Tween 80 in water) administered at 10 ml/kg; bid; p.o. Tumor volumes were estimated using the largest diameter (L) and a diameter perpendicular to this (W) according to (L × W²) * π/6. Data are presented as means ± SEM. Statistical analysis was performed using one-way ANOVA with either post hoc Dunnet's test for comparison of treatment versus vehicle control group (*P < 0.05) or the post-hoc Tukey test for intra group analysis (^{$!#}P < 0.05).

TABLE 5
MIS-59FVA-12 Day 14 Values

| | | Tumor response | | Host response | | |
|---|---|---|---|---|---|---|
| | | | ΔTumor | | Δbody | |
| | Dose, schedule, | | volume | Δbody | weight | Survival |
| Compound | route | T/C (%) | (mm³) | weight (g) | (%) | (alive/total) |
| Vehicle | 10 ml/kg, bid | 100 | 90.1 ± 16.4 | −0.8 ± 0.3 | −2.8 ± 1.0 | 7/7 |
| | (q12h), p.o. | | | | | |
| D | 300 mg/kg, bid | 40.3* | 36.3 ± 17.0 | −0.7 ± 0.3 | −2.4 ± 1.0 | 7/7 |
| | (q12h), p.o. | | | | | |
| C | 30 mg/kg, qd | 4.0%* | 3.6 ± 7.6 | −0.9 ± 0.3 | −3.2 ± 1.0 | 7/7 |
| | (q24h), p.o. | | | | | |
| C + D | 30 mg/kg, qd + 300 | Regression | −12.4 ± 8.1 | −1.2 ± 0.5 | −4.3 ± 1.7 | 7/7 |
| | mg/kg, bid, p.o. | 9.2%* | | | | |

Tumors were established in male SCID mice by subcutaneous inoculation of human prostate VCap cells (5 × 10⁶ cells/100 μl HBSS + matrigel 1:1 mixture). On the day cell inoculation animals were chemically castrated by intramuscular injection of 15 μl of Lucrin (3.73 mg/ml). Forty eight days after cell inoculation, treatment was started on day 0 with compounds D, (Abiraterone Acetate, AA), C, and combination of C + D at the doses and schedules listed above (mg/kg). The compounds were dosed orally and compared with control vehicle (0.45% methyl cellulose, 0.1% Tween 80 in water) administered at 10 ml/kg; bid; p.o. Tumor volumes were estimated using the largest diameter (L) and a diameter perpendicular to this (W) according to (L × W²) * π/6. Data are presented as means ± SEM. Statistical analysis was performed using one-way ANOVA with either post hoc Dunnet's test for comparison of treatment versus vehicle control group (*P < 0.05) or the post-hoc Tukey test for intra-group analysis which showed no significant differences between the 3 drug treated groups (P > 0.05).

TABLE 6
MIS-59FVA-12 Day 23 Values

| | | Tumor response | | Host response | | |
|---|---|---|---|---|---|---|
| | | | ΔTumor | | Δbody | |
| | Dose, schedule, | | volume | Δbody | weight | Survival |
| Compound | route | T/C (%) | (mm³) | weight (g) | (%) | (alive/total) |
| Vehicle | 10 ml/kg, bid | 100 | 206.4 ± 23.5 | 1.0 ± 0.4 | 4.1 ± 1.6 | 7/7 |
| | (q12h), p.o. | | | | | |
| D | 300 mg/kg, bid | 38.7*^{;#} | 79.8 ± 20.1 | 0.2 ± 0.1 | 0.6 ± 0.5 | 7/7 |
| | (q12h), p.o. | | | | | |
| C | 30 mg/kg, qd | 12.4* | 25.6 ± 16.4 | 0.7 ± 0.3 | 3.0 ± 1.1 | 7/7 |
| | (q24h), p.o. | | | | | |
| C + D | 30 mg/kg, qd + 300 | Regression | −12.26 ± 8.84 | 0.5 ± 0.5 | 2.3 ± 2.0 | 7/7 |
| | mg/kg, bid, p.o. | 9.0*^{;#} | | | | |

Tumors were established in male SCID mice by subcutaneous inoculation of human prostate VCap cells (5 × 10⁶ cells/100 μl HBSS + matrigel 1:1 mixture). On the day cell inoculation animals were chemically castrated by intramuscular injection of 15 μl of Lucrin (3.73 mg/ml). Forty eight days after cell inoculation, treatment was started on day 0 with compounds D, (Abiraterone Acetate, AA), C, and combination of C + D at the doses and schedules listed above (mg/kg). The compounds were dosed orally and compared with control vehicle (0.45% methyl cellulose, 0.1% Tween 80 in water) administered at 10 ml/kg; bid; p.o. Tumor volumes were estimated using the largest diameter (L) and a diameter perpendicular to this (W) according to (L × W²) * π/6. Data are presented as means ± SEM. Statistical analysis was performed using one-way ANOVA with either post hoc Dunnet's test for comparison of treatment versus vehicle control group (*P < 0.05) or the post-hoc Tukey test for intra-group analysis showed significant difference between the combination group and CFG920 alone group (*P < 0.05).

TABLE 7
MIS-43P-12

| Days of treatment | | 0 | 4 | 7 | 11 | 14 | 18 |
|---|---|---|---|---|---|---|---|
| Vehicle | Average | 170 | 223 | 293 | 401 | 520 | 646 |
| | Stdev | 45 | 68 | 98 | 137 | 195 | 257 |
| | Sem | 17 | 26 | 37 | 52 | 74 | 97 |
| | Delta avg | 0 | 52 | 123 | 231 | 349 | 475 |
| | T/C [%] | 100 | 100 | 100 | 100 | 100 | 100 |
| Compound C, | Average | 170 | 199 | 258 | 369 | 432 | 546 |
| 30 mg/kg, qd, po | Stdev | 45 | 54 | 64 | 79 | 140 | 170 |
| | Sem | 17 | 20 | 24 | 30 | 53 | 70 |
| | Delta avg | 0 | 29 | 88 | 198 | 261 | 375 |
| | T/C [%] | 100 | 56 | 71 | 86 | 75 | 79 |
| Compound C/ | Average | 171 | 191 | 224 | 275 | 347 | 395 |
| Compound D, | Stdev | 44 | 50 | 82 | 75 | 128 | 132 |
| 30/300 | Sem | 17 | 19 | 31 | 28 | 48 | 50 |
| mg/kg/mg/kg, | Delta avg | 0 | 20 | 53 | 104 | 176 | 223 |
| qd/bid, po | T/C [%] | 100 | 39 | 43 | 45 | 50 | 47 |
| Compound D, | Average | 174 | 217 | 248 | 372 | 375 | 491 |
| 300 mg/kg, bid, po | Stdev | 31 | 73 | 98 | 101 | 121 | 142 |
| | Sem | 12 | 28 | 37 | 38 | 46 | 54 |
| | Delta avg | 0 | 43 | 73 | 197 | 200 | 317 |
| | T/C [%] | 100 | 82 | 60 | 85 | 57 | 67 |

| Days of treatment | | 0 | 4 | 9 | 14 | 18 |
|---|---|---|---|---|---|---|
| Compound E, | Average | 164 | 224 | 312 | 450 | 554 |
| 100 mg/kg, bid, po | Stdev | 24 | 71 | 87 | 178 | 193 |
| | Sem | 9 | 27 | 33 | 67 | 73 |
| | Delta avg | 0 | 60 | 148 | 286 | 390 |
| | T/C [%] | 100 | 115 | 84 | 82 | 82 |

### Example 4

### Clinical Trial

A clinical study using (a) a phosphatidylinositol 3-kinase inhibitor that is either COMPOUND A or its monotosylate salt or COMPOUND C or its hydrochloride salt, in combination with (b) 1-(2-Chloro-pyridin-4-yl)-3-(4-methyl-pyridin-3-yl)-imidazolidin-2-one or a pharmaceutically acceptable salt thereof, and (c) prednisone for treatment of patients with castration-resistant prostate cancer after failure of abiraterone acetate therapy is investigated.

An open-label, unblinded study of the combination comprising (a) either COMPOUND A or its monotosylate salt or COMPOUND C or its hydrochloride salt, (b) 1-(2-Chloro-pyridin-4-yl)-3-(4-methyl-pyridin-3-yl)-imidazolidin-2-one or a pharmaceutically acceptable salt thereof, and (c) prednisone is conducted in patients who are diagnosed with a castration-resistant prostate cancer and after failure of abiraterone acetate therapy. In the first phase, a dose-escalation study is conducted to determine the maximal tolerated dose (MTD) and/or recommended dose for expansion (RDE) of both (a) COMPOUND A or its monotosylate salt in combination with 1-(2-Chloro-pyridin-4-yl)-3-(4-methyl-pyridin-3-yl)-imidazolidin-2-one and prednisone (hereinafter referred to as “COMPOUND A Arm 1”), and (b) COMPOUND C or its monohydrochloride salt in combination with 1-(2-Chloro-pyridin-4-yl)-3-(4-methyl-pyridin-3-yl)-imidazolidin-2-one and prednisone (hereinafter referred to as “COMPOUND C Arm 1”) in patients diagnosed with castration-resistant prostate cancer after failure of abiraterone acetate therapy. Approximately 15 to 25 patients are enrolled in the COMPOUND A Arm 1, and approximately 10 to 20 patients are enrolled in the COMPOUND C Arm 1 in the first phase. In the second phase, a dose expansion phase is conducted to investigate the antitumor activity of both (a) COMPOUND A or its monotosylate salt in combination with 1-(2-Chloro-pyridin-4-yl)-3-(4-methyl-pyridin-3-yl)-imidazolidin-2-one and prednisone (hereinafter referred to as “COMPOUND A Arm 2”), and (b) COMPOUND C or its monohydrochloride salt in combination with 1-(2-Chloro-pyridin-4-yl)-3-(4-methyl-pyridin-3-yl)-imidazolidin-2-one and prednisone (hereinafter referred to as “COMPOUND C Arm 2”) in patients diagnosed with castration-resistant prostate cancer after failure of treatment with abiraterone acetate therapy. Approximately 35 to 45 patients are enrolled in the COMPOUND A Arm 2, and approximately 35 to 45 patients are enrolled in the COMPOUND C Arm 2 in the second phase. Patients are treated until disease progression, unacceptable toxicity, death, or discontinuation from the study treatment due to any other reason.

For the clinical study, “failure of abiraterone acetate therapy” is defined as progression of the castration-resistant prostate cancer after treatment with abiraterone acetate therapy alone or abiraterone acetate therapy in combination with prednisone or corticosteroids administered by topical application, inhalation, eye drops, or local injection. In the first phase, patients have a documented progressive disease to prior abiraterone acetate therapy according to Prostate Cancer Working Group 2 (PCWG2) criteria according to at least one of the following criteria: (a) patient who progressed based solely upon rising levels of the serum marker Prostate-Specific Antigen (PSA), wherein the sequence of rising values occurs on three (3) consecutive occasions of at least 1 week intervals and having 5.0 ng/mL minimum level for entry into the study, (b) patient who manifest disease progression per Response Evaluation Criteria in Solid Tumors (“RECIST”) independent of PSA level, and/or (c) patient with bone only progression according to PCWG2. In the second phase, patients must have had a prior Prostate-Specific Antigen response followed by documented Prostate-Specific Antigen (“PSA”) progression on abiraterone acetate monotherapy. “PSA progression” is defined as a sequence of rising levels of PSA on three (3) consecutive occasions of at least 1 week intervals and having 5.0 ng/mL minimum level for entry into the study,

Potential patients are screened within 7 to 28 day days prior to treatment start. The following inclusion and exclusion criteria define those patients eligible for the study:

Inclusion Criteria:

Patients eligible for this study are those satisfying all of the following criteria:
Adult males ≧18 years old
Eastern Cooperative Oncology Group Performance Status ≦2
Patient having a castrate level of testosterone (<=50 ng/dL or 1.7 nmol/L). Castrate status must be maintained by continued Gonadotropin-releasing hormone (GnRH) analogues unless patient has undergone surgical orchiectomy).
Histologically or cytologically confirmed diagnosis of advanced or metastatic castration resistant prostate cancer.
Advanced or metastatic castration-resistant prostate cancer progression after abiraterone acetate failure
Patients having no more than 2 lines of prior chemotherapies including cytotoxic agents (i.e. docetaxel)
Discontinuation of all anti-androgen, anti-neoplastic or investigational treatment >=4 weeks (6 weeks for bicalutamide). Patient has adequate bone marrow and organ function as defined by the following laboratory values:

Additional inclusion criteria for First Phase (dose escalation part) only):
Patient having documented progressive disease to the previous line of therapy according to PCWG2 criteria:
   Patient who progressed based solely on Prostate-Specific Antigen (PSA) rising levels, should have had a sequence of rising values on 3 consecutive occasions of at least 1 week intervals and should have 5.0 ng/mL minimum level for entry.
   Patients who manifested disease progression per RECIST are eligible independent of PSA.
   Patients with bone only progression according to PCWG2.

Additional inclusion criteria for Second Phase (dose expansion part) only:
Patient having a prior Prostate-Specific Antigen (PSA) response, followed by documented PSA progression on abiraterone acetate therapy.
“PSA progression” is defined as a sequence of rising values on 3 consecutive occasions of at least 1 week intervals and should have 5.0 ng/mL minimum level for entry.
Abiraterone Acetate is the last treatment prior to enrollment in the dose expansion part of the study and no other anticancer therapy is allowed between Abiraterone Acetate and study entry.

Exclusion Criteria:

Patients eligible for this study are those that do not meet any of the following criteria:
Previous treatment with PI3K pathway inhibitors (e.g. PI3K, AKT, mTOR inhibitor), ketoconazole, or other CYP17 inhibitors (exception of AA), or MDV3100.
Patient having active uncontrolled or symptomatic Central Nervous System

(CNS) metastases. A patient with controlled and asymptomatic CNS metastases may participate in this trial. As such, the patient must have completed any prior treatment for CNS metastases >28 days (including radiotherapy and/or surgery) prior to start of treatment in this study and should not be receiving chronic corticosteroid therapy for the CNS metastases.
Inadequately controlled hypertension (e.g. systolic blood pressure >=160 mmHg or diastolic blood pressure >=95 mmHg)
Patient having a QT interval corrected for heart rate using Fridericia's formula

(QTcF) >480 msec on the screening Electrocardiogram (using the QT interval corrected for heart rate using Fridericia's formula), has a short/long QT syndrome, or history of QT prolongation/Torsades de Pointes
Patient is currently receiving treatment with medication that has a known risk to prolong the QT interval or inducing Torsades de Pointes, and the treatment cannot be discontinued or switched to a different medication prior to treatment start.
Patient is currently receiving increasing or chronic treatment (>5 days) with corticosteroids or another immunosuppressive agent, as chronic administration of corticosteroids (>5 days) can induce CYP3A4. The following corticosteroids are permitted:
e. Prednisone 5 mg BID as part of the study treatment;
f. Topical applications (e.g., rash), inhaled sprays (e.g., obstructive airways diseases), eye drops or local injections (e.g., intra-articular)
Patient having impairment of gastrointestinal (GI) function or GI disease that may significantly alter the absorption of study drugs (e.g., ulcerative diseases, uncontrolled nausea, vomiting, diarrhea, malabsorption syndrome, or small bowel resection)
Patient having a medically documented history of or active major depressive episode, bipolar disorder (I or II), obsessive-compulsive disorder, schizophrenia, a history of suicidal attempt or ideation, or homicidal ideation (e.g. risk of doing harm to self or others) and/or (a) patient has CTCAE grade 3 anxiety, (b) Patient has a Generalized Anxiety Disorder 7-item scale (GAD-7) mood scale score ≧15, or (c) Patient has a score ≧12 on the Patient Health Questionaire-9 item (PHQ-9) questionnaire.
Patients who has experienced dose reductions and/or treatment interruptions due to 1-(2-Chloro-pyridin-4-yl)-3-(4-methyl-pyridin-3-yl)-imidazolidin-2-one related toxicities (i.e. serious adverse events, adverse events, liver toxicities during 1-(2-Chloro-pyridin-4-yl)-3-(4-methyl-pyridin-3-yl)-imidazolidin-2-one treatment

Additional exclusion criteria for Second Phase (dose expansion part) only:
Patients who has received any anticancer systemic therapy between abiraterone acetate and study entry
   During the screening, the patient's diagnosis and extent of cancer (including staging, histology/cytology and sites of disease at study entry, PSA history and PSA level), demography, medical history, prior antineoplastic therapies, all medications and significant non-drug therapies taken within 28 days before first dose, and other physical characteristics (e.g, vital signs, height, weight, physical examination, ECOG, laboratory evaluations of blood, ECG, cardiac imaging, CT/MRI and bone scan assessments of tumors and additional radiological assessments if clinically indicated, and mood) are assessed.

In the First Phase (Dose Escalation) of the clinical trial, the primary objective is to determine (a) the MTD and/or RDE of COMPOUND A or its monotosylate salt in combination with 1-(2-Chloro-pyridin-4-yl)-3-(4-methyl-pyridin-3-yl)-imidazolidin-2-one and (b) the MTD and/or RDE of COMPOUND C or its monohydrochloride salt in combination with 1-(2-Chloro-pyridin-4-yl)-3-(4-methyl-pyridin-3-yl)-imidazolidin-2-one. A secondary objective is to determine the percent change from baseline in PSA at Week 12 as well as the best percentage change from baseline at any time as calculated using water fall plots. The proportion of patients with PSA decrease from baseline of at least 30% at Week 12 or later is calculated for patients of the first phase.

In the COMPOUND A Arm 1 group, patients are provisionally administered each of the following: (a) 200 mg of COMPOUND A, per oral, twice daily (B.I.D.) for a total daily dose of 400 mg; (b) 100-1000 mg 1-(2-Chloro-pyridin-4-yl)-3-(4-methyl-pyridin-3-yl)-imidazolidin-2-one, per oral, once daily (Q.D.); and (c) 5 mg prednisone, per oral, twice daily (B.I.D.) for a total daily dose of 10 mg. In the COMPOUND C Arm 1 group, patients are provisionally administered each of the following: (a) 60 mg of COMPOUND C, per oral, once daily (Q.D.), (b) 100-1000 mg 1-(2-Chloro-pyridin-4-yl)-3-(4-methyl-pyridin-3-yl)-imidazolidin-2-one, per oral, once daily (Q.D.); and (c) 5 mg prednisone, per oral, twice daily (B.I.D.) for a total daily dose of 10 mg. COMPOUND A may be administered in the form of its free base or its monotosylate salt form. A complete treatment cycle is defined as 35 calendar days consisting of (a) a 7-day run-in period during which 1-(2-Chloro-pyridin-4-yl)-3-(4-methyl-pyridin-3-yl)-imidazolidin-2-one or its pharmaceutically acceptable salt is given once daily and prednisone is given twice daily and (b) 28 days combination treatment period during which COMPOUND A or its monotosylate salt and prednisone are given twice daily and 1-(2-Chloro-pyridin-4-yl)-3-(4-methyl-pyridin-3-yl)-imidazolidin-2-one or its pharmaceutically acceptable salt is given once daily. Combination treatment with COMPOUND A or its monotosylate salt begins on Day 8 of Cycle 1. The last day of the first treatment cycle is Day 35. All subsequent treatment cycles for COMPOUND A Arm 1 consist of 28 calendar days during which COMPOUND A or its monotosylate salt and prednisone are given twice daily and 1-(2-Chloro-pyridin-4-yl)-3-(4-methyl-pyridin-3-yl)-imidazolidin-2-one is given once daily.

In the COMPOUND C Arm 1 group, patients are provisionally administered each of the following: (a) 60 mg of COMPOUND C, per oral, once daily (Q.D.); (b) 100-1000 mg 1-(2-Chloro-pyridin-4-yl)-3-(4-methyl-pyridin-3-yl)-imidazolidin-2-one, per oral, once daily (Q.D.); and (c) 5 mg prednisone, per oral, twice daily (B.I.D.) for a total daily dose of 10 mg. COMPOUND C may be administered in the form of its free base or its monohydrochloride salt form. A complete treatment cycle consists of 28 calendar days during which COMPOUND C or its monohydrochloride salt and 1-(2-Chloro-pyridin-4-yl)-3-(4-methyl-pyridin-3-yl)-imidazolidin-2-one are given once daily and prednisone is given twice daily. The last day of the complete treatment cycle is Day 28.

A cohort of approximately 3-6 patients is treated at the next dose level. Each cohort is consisting of newly enrolled patients. To account for the potential of drop-outs during the first cycle of treatment (e.g., due to early disease progression), up to up to 2 additional patients may be enrolled, if these additional patients can be treated within 14 days after the third patient is first dosed with the COMPOUND A or its monotosylate salt. The dose levels for the dose-escalation phase of the clinical study are defined as follows for the COMPOUND A Arm 1 Group:

| | | 1-(2-Chloro-pyridin-4-yl)- | |
|---|---|---|---|
| Dose | COMPOUND | 3-(4-methyl-pyridin-3-yl)- | |
| level | A | imidazolidin-2-one | Prednisone |
| Dose | 200 mg BID | 100-1000 mg (QD) | 5 mg BID (2 × 5 |
| Level 1 | (2 × 200 mg | | mg for a total |
| | for total of | | of 10 mg a day) |
| | 400 mg a day) | | |
| Dose | 300 mg BID | 100-1000 mg (QD) | 5 mg BID (2 × 5 |
| Level 2 | (2 × 300 mg | | mg for a total |
| | for total of | | of 10 mg a day) |
| | 600 mg a day) | | |
| Dose | 400 mg BID | 100-1000 mg (QD) | 5 mg BID (2 × 5 |
| Level 3 | (2 × 400 mg | | mg for a total |
| | for total of | | of 10 mg a day) |
| | 800 mg a day) | | |

Further, the dose levels for the dose-escalation phase of the clinical study are defined as follows for the COMPOUND C Arm 1 Group:

| | | 1-(2-Chloro-pyridin-4-yl)- | |
|---|---|---|---|
| Dose | COMPOUND | 3-(4-methyl-pyridin-3-yl)- | |
| level | C | imidazolidin-2-one | Prednisone |
| Dose | 60 mg (QD) | 100-1000 mg (QD) | 5 mg BID (2 × 5 |
| Level 1 | | | mg for a total of |
| | | | 10 mg a day) |
| Dose | 80 mg (QD) | 100-1000 mg (QD) | 5 mg BID (2 × 5 |
| Level 2 | | | mg for a total of |
| | | | 10 mg a day) |
| Dose | 100 mg (QD) | 100-1000 mg (QD) | 5 mg BID (2 × 5 |
| Level 3 | | | mg for a total of |
| | | | 10 mg a day) |

In the first phase, the individual dose is dependent on the current dose level at which the patient enters the study. A five-parameter adaptive Bayesian logistic regression model guided by escalation with overdose control (EWOC) principle is used for dose level selection and for determination of the MTD and/or RDE of the COMPOUND A combination treatment and the COMPOUND C combination treatment. Patients are considered evaluable for the dose-determining set if the patient experiences a dose-limiting toxicity (DLT) during cycle 1, or meets the minimum treatment exposure and safety evaluations required as defined by the following criteria:
(a) Patients in the COMPOUND A Arm 1 group: The minimum exposure criterion is at least 21 of the 28 full daily planned doses of 1-(2-Chloro-pyridin-4-yl)-3-(4-methyl-pyridin-3-yl)-imidazolidin-2-one and COMPOUND A or its monotosylate salt between Day 8 and Day 35 of Cycle 1.
(b) Patients in the COMPOUND C Arm 1 group: The minimum exposure crtierion is at least 21 of the 28 full daily planned doses of 1-(2-Chloro-pyridin-4-yl)-3-(4-methyl-pyridin-3-yl)-imidazolidin-2-one and COMPOUND C or its monohydrocloride salt between Day 1 and Day 28 of Cycle 1.
(c) Patients should have completed the required safety evaluations during Cycle 1 to an extent that the investigator or physician can determine that no unacceptable toxicities occurred in Cycle 1 for this dosing cohort.

To determine the dose level for the next cohort in the dose-escalation, the available toxicity information (including dose limiting toxicities and adverse events that are not dose limiting toxicities), pharmacokinetics, pharmacodynamics, efficacy information and recommendations from the five-parameter adaptive Bayesian logistic regression model (BLRM) are evaluated. A 5 parameter BLRM for each combination treatment is fitted on Cycle 1 dose-limiting toxciity data (i.e., absence or presence of DLT) accumulated throughout the dose-escalation to model the dose-toxicity relationship of 1-(2-Chloro-pyridin-4-yl)-3-(4-methyl-pyridin-3-yl)-imidazolidin-2-one and either COMPOUND A or COMPOUND C when given in combination. The dose for the next cohort is not exceeding the maximum dose allowed by the five-parameter adaptive Bayesian logistic regression model. After completion of cycle 1 of each cohort, the BLRM is used to make recommendations about the next dose level, with the following exceptions:
If 2 of the first 3 patients in a cohort experience a dose limiting toxicity (DLT), further enrollment to that cohort is suspended. The BLRM is updated with this new information before additional patients are enrolled to the study.
If a decision is made to escalate to a higher dose level, but one or more additional patient(s) are treated at the preceding dose level experiences a dose limiting toxicity (DLT), in cycle 1, then the BRLM is updated before any additional patient is enrolled to the higher dose level.
   Dose escalation is continued until identification of the MTD and/or RDE.

A dose-limiting toxicity (DLT) is defined as an adverse event or abnormal laboratory value assessed as unrelated to disease, disease progression, inter-current illness, or concomitant medications that occurs within the first cycle and meets any of the criteria included below. National Cancer Institute Common Terminology Criteria for Adverse events (NCI CTCAE) version 4.03, which is hereby incorporated by reference in its entirety, will be used for all grading. For the purpose of dose-escalation decisions, the following DLTs are considered for these drug combinations:

TABLE 8

| TOXICITY | ANY OF THE FOLLOWING CRITERIA |
|---|---|
| Hematologic | CTCAE grade 4 neutropenia |
| | CTCAE grade 4 thrombocytopenia |
| | Febrile neutropenia (ANC <1.0 × 10⁹/L, fever ≧38.3° C.) |
| | CTCAE Grade ≧3. |
| Renal | ≧CTCAE grade 3 serum creatinine |
| Hepatic | Total bilirubin ≧2.0 × ULN to ≦3.0 × ULN for |
| | >7 consecutive days. |
| | AST or ALT CTCAE Grade ≧3 in conjunction with blood |
| | bilirubin CTCAE Grade ≧2 of any duration |
| | ≧CTCAE grade 3 total bilirubin |
| | CTCAE grade 3 AST or ALT for >7 consecutive days |
| | CTCAE grade 3 GGT elevation in conjunction with CTCAE |
| | Grade ≧3. AST and/or ALT |
| | CTCAE grade 4 AST or ALT |
| | CTCAE grade 4 GGT elevation in conjunction with CTCAE |
| | Grade ≧3. AST and/or ALT |
| Endocrine | ≧Grade 3 hyperglycemia (>13.89 mmol/L or 250 mg/dL |
| (Hyperglycemia is NOT graded | FPG) (confirmed with a repeat FPG within 24 hours) |
| per CTCAE v4.03) | |
| Metabolic/Laboratory | CTCAE grade 3 asymptomatic amylase and/or lipase |
| | >7 consecutive days |
| | CTCAE grade 4 asymptomatic amylase and/or lipase |
| Pancreatitis | ≧CTCAE grade 2 |
| Cardiac | Cardiac toxicity ≧CTCAE grade 3 or cardiac event that is |
| | symptomatic or requires medical intervention |
| | Clinical signs of cardiac disease, such as unstable angina |
| | or myocardial infarction, or Troponin ≧CTCAE grade 3 |
| Mood alteration | CTCAE grade 2 mood alteration that does not resolve to |
| | ≦grade 1 within 14 days despite medical treatment (for |
| | Anxiety only, if worsened from baseline) |
| | ≧CTCAE grade 3 mood alteration |
| | CTCAE Grade 3 rash for >7 consecutive days despite skin |
| | toxicity treatment |
| | CTCAE Grade 4 rash |
| Other adverse events | ≧CTCAE grade 3 adverse events (excluding ≧CTCAE |
| | grade 3 elevations in alkaline phosphatase) |
| | ≧CTCAE grade 3 vomiting/nausea ≧48 hrs, despite the |
| | use of anti-emetic therapy |
| | ≧CTCAE grade 3 diarrhea ≧48 hrs, despite the use of |
| | anti-diarrheal therapy |
| | CTCAE grade 3 fatigue (asthenia) for >7 consecutive days |
| | CTCAE grade 4 fatigue (asthenia) |
| | CTCAE grade 3 stomatitis/mucositis ≧3 days, despite the |
| | use of topical corticosteroids and analgesic mouth |
| | treatments |
| | CTCAE grade 4 stomatitis/mucositis |

Further DLTs include (a) the inability to take 75% or more of the planned daily doses from Days 8-35 in Cycle 1 due to treatment-related adverse events in the COMPOUND A Arm 1 (i.e. >7 days of partial or no dose of COMPOUND A or its monotosylate salt or 1-(2-Chloro-pyridin-4-yl)-3-(4-methyl-pyridin-3-yl)-imidazolidin-2-one in Days 8-35); or (b) the inability to take 75% or more of the planned daily doses from Days 8-35 in Cycle 1 due to treatment-related AEs in the COMPOUND C Arm 1 (i.e. >7 days of partial or no dose of COMPOUND C or its monohydrochloride salt or 1-(2-Chloro-pyridin-4-yl)-3-(4-methyl-pyridin-3-yl)-imidazolidin-2-one in Days 8-35).

“MTD” is defined as the highest drug dosage that does not cause medically unacceptable dose-limiting toxicities in more than 35% of the treated patients during the first cycle of treatment. Typically the MTD is a tested dose with a maximum probability of targeted toxicity (DLT rate between 16%-35%). “RDE” is the dose that is recommended for further use in the expansion part of this clinical study. The RDE may be determined to be the same as the MTD. The declared RDE may also be lower than the MTD if the evolving safety profile (long term or overall) along with other assessments such as PK suggest a better safety profile without substantial loss of benefit in exposure/activity.

In the Second Phase (Dose Expansion) of the clinical trial, the primary objective is to assess the anti-tumor activity of the combinations (i.e., 1-(2-Chloro-pyridin-4-yl)-3-(4-methyl-pyridin-3-yl)-imidazolidin-2-one+COMPOUND A or its monotosylate salt and abiraterone acetate+COMPOUND C or its monohydrochloride salt) in patients diagnosed with castration-resistant prostate cancer patients and after failure of 1-(2-Chloro-pyridin-4-yl)-3-(4-methyl-pyridin-3-yl)-imidazolidin-2-one monotherapy. A further variable objective is also the proportion of patients in the FAS, with a PSA decrease from baseline of at least 30% at Week 12 or later. Patients are treated with the COMPOUND A Combination (COMPOUND A Arm 2 Group) or the COMPOUND C Combination (COMPOUND C Arm 2 Group) at the MTD or RDE determined in the First Phase. At the time point when the second phase starts, patients from earlier cohorts can be dose-escalated up to the MTD if all of the following criteria are met:
(a) Patient has been treated for least 4 months (4 cycles);
(b) No treatment related toxicity has occurred with the current dose level, resulting in treatment reduction or interruption or discontinuation in the previous 2 cycles.
   These patients repeat the schedule of safety assessments as done for cycle 1 (starting at Day 1) for 1 cycle. Dose escalation is done only after the patient completes the ongoing cycle.

All treatment cycles in the COMPOUND A Arm 2 group consist of 28 calendar days during which COMPOUND A or its monotosylate salt and prednisone are given to each patient twice daily an 1-(2-Chloro-pyridin-4-yl)-3-(4-methyl-pyridin-3-yl)-imidazolidin-2-one is given once daily. All treatment cycles in the COMPOUND C Arm 2 group consist of 28 calendar days during which COMPOUND C or its monohydrocloride salt and 1-(2-Chloro-pyridin-4-yl)-3-(4-methyl-pyridin-3-yl)-imidazolidin-2-one are given once daily and prednisone is given twice daily. The last day of a complete treatment cycle is Day 28.

Patients are treated until disease progression, unacceptable toxicity, death or discontinuation from the study treatment due to any other reason. No patients are replaced during the second phase.

Efficacy

In the first and second phase, efficacy may be assessed by decline change in PSA level, time to progression of PSA, radiological progression-free survival, radiological response and/or overall survival.

PSA is measured regularly to calculate PSA decline and time to PSA progression. PSA progression is defined as per PCWG2 as follows:
(a) For patients with decline in PSA levels from baseline: Time from start of therapy to first PSA increase that is ≧25% above the nadir and ≧2 ng/mL above the nadir, and which is confirmed by a second value 3 or more weeks later (ie, a confirmed rising trend) sequentially.
(b) For patients with no decline in PSA levels from baseline: Time from start of therapy to first PSA increase that is ≧25% above baseline and ≧2 ng/mL above baseline after 12 weeks.

Time to PSA progression (TTpP) is calculated and defined according to PCWG2 as follows:
(a) for patients whose PSA has initially decreased, TTpP is defined as the time from start of treatment to the first PSA increase that is ≧25% above the nadir and ≧2 ng/mL above the nadir and which is confirmed by a second value 3 or more weeks later sequentially.
(b) for patients whose PSA has not decreased, TTpP is defined as the time from start of treatment to the first PSA increase that is ≧25% above the nadir and ≧2 ng/mL above the baseline.
   Increase in PSA within the first 12 weeks of treatment will not be considered as progression (unless unequivocal sign of progression).

Radiological progression-free survival is defined per PCWG2 as the time from start of treatment until progression (based on local assessment) or death from any cause. Progression is defined as the occurrence of either tumor progression in soft tissue according to RECIST 1.1 or progression in bone. Progression on Bone scans is defined as the appearance of at least 2 new lesions as compared to the previous scan. For bone scans, progression at the first assessment (week 12) is defined as at least 2 new lesions, is confirmed by a subsequent scan 6 or more weeks later showing at least 2 additional new bone lesions as compared to the first assessment scan as per PCWG2. For subsequent evaluations, according to PCWG2, bone progression is defined as at least 2 new lesions as compared to the previous scan, should be confirmed by a subsequent scan 6 or more weeks later. Progression on CT scan at first assessment are confirmed by a subsequent scan 6 or more weeks later as per PCWG2. Progression on CT scans (soft tissues) and progression on bone scans are analyzed separately.

Radiological response refers to the best overall response according to RECIST 1.1.

Overall survival is defined as the time from start of treatment to the date of death due to any cause.

Efficacy may be preliminarily assessed based upon interim study data as known to one of ordinary skill in the art.

Follow-Up

All patients have safety evaluations for 30 days after last dose of study treatment.

If a patient did not discontinue study treatment due to disease progression, death, start of new anti-neoplastic therapies, lost to follow-up, or withdrawal of consent to efficacy follow-up, then tumor assessments are continuing to be performed every 12 weeks until the start of new anti-cancer therapy, disease progression, death, lost to follow-up, or withdrawal of consent to efficacy follow-up.

All patients are followed for survival status every 3 months regardless of treatment discontinuation reason until death, lost to follow-up, or withdrawal of consent to survival follow-up. Additional survival assessments may be performed if a survival update is required for an interim assessment.

1. A combination comprising (a) a phosphatidylinositol 3-kinase inhibitor selected from the group consisting of the compound of formula (I), 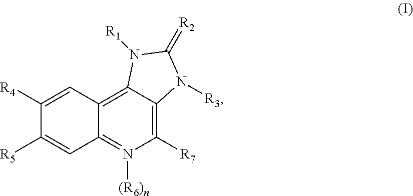
wherein
R₁ is naphthyl or phenyl wherein said phenyl is substituted by one or two substituents independently selected from the group consisting of Halogen; lower alkyl unsubstituted or substituted by halogen, cyano, imidazolyl or triazolyl; cycloalkyl; amino substituted by one or two substituents independently selected from the group consisting of lower alkyl, lower alkyl sulfonyl, lower alkoxy and lower alkoxy lower alkylamino; piperazinyl unsubstituted or substituted by one or two substituents independently selected from the group consisting of lower alkyl and lower alkyl sulfonyl; 2-oxo-pyrrolidinyl; lower alkoxy lower alkyl; imidazolyl; pyrazolyl; and triazolyl;
R₂ is O or S;
R₃ is lower alkyl;
R₄ is pyridyl unsubstituted or substituted by halogen, cyano, lower alkyl, lower alkoxy or piperazinyl unsubstituted or substituted by lower alkyl; pyrimidinyl unsubstituted or substituted by lower alkoxy; quinolinyl unsubstituted or substituted by halogen; quinoxalinyl; or phenyl substituted with alkoxy
R₅ is hydrogen or halogen;
n is 0 or 1;
R₆ is oxido;
with the proviso that if n=1, the N-atom bearing the radical R₆ has a positive charge;
R₇ is hydrogen or amino;
or compound of formula (II), 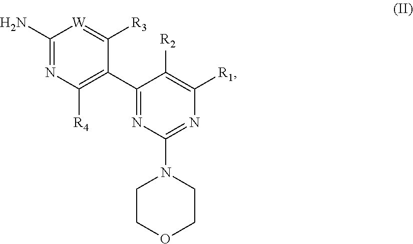
wherein W is CR_{w} or N, wherein
R_{w} is selected from the group consisting of:
(1) hydrogen,
(2) cyano,
(3) halogen,
(4) methyl,
5) trifluoromethyl,
(6) sulfonamide;
R₁ is selected from the group consisting of:
(1) hydrogen,
(2) cyano,
(3) nitro,
(4) halogen,
(5) substituted and unsubstituted alkyl,
(6) substituted and unsubstituted alkenyl,
(7) substituted and unsubstituted alkynyl,
(8) substituted and unsubstituted aryl,
(9) substituted and unsubstituted heteroaryl,
(10) substituted and unsubstituted heterocyclyl,
(11) substituted and unsubstituted cycloalkyl,
(12) —COR₁ₐ,
(13) —CO₂R₁ₐ,
(14) —CONR₁ₐR_{1b},
(15) —NR₁ₐR_{1b},
(16) —NR₁ₐCOR_{1b},
(17) —NR₁ₐSO₂R_{1b},
(18) —OCOR₁ₐ,
(19) —OR₁ₐ,
(20) —SR₁ₐ,
(21) —SOR₁ₐ,
(23) —SO₂NR_{Ia}R_{1b} wherein
R₁ₐ, and R_{1b} are independently selected from the group consisting of:
(a) hydrogen,
(b) substituted or unsubstituted alkyl,
(c) substituted and unsubstituted aryl,
(d) substituted and unsubstituted heteroaryl,
(e) substituted and unsubstituted heterocyclyl, and
(f) substituted and unsubstituted cycloalkyl;
R₂ is selected from the group consisting of:
(1) hydrogen,
(2) cyano,
(3) nitro,
(4) halogen,
(5) hydroxy,
(6) amino,
(7) substituted and unsubstituted alkyl,
(8) —COR₂ₐ, and
(9) —NR₂ₐCOR_{2b}, wherein
R₂ₐ, and R_{2b} are independently selected from the group consisting of:
(a) hydrogen, and
(b) substituted or unsubstituted alkyl;
R₃ is selected from the group consisting of:
(1) hydrogen,
(2) cyano,
(3) nitro,
(4) halogen,
(5) substituted and unsubstituted alkyl,
(6) substituted and unsubstituted alkenyl,
(7) substituted and unsubstituted alkynyl,
(8) substituted and unsubstituted aryl,
(9) substituted and unsubstituted heteroaryl,
(10) substituted and unsubstituted heterocyclyl,
(11) substituted and unsubstituted cycloalkyl,
(12) —COR₃ₐ,
(14) —NR₃ₐR_{3b}
(13) —NR₃ₐCOR_{3b},
(15) —NR₃ₐSO₂R_{3b},
(16) —OR₃ₐ,
(17) —SR₃ₐ,
(18) —SOR₃ₐ,
(19) —SO₂R₃ₐ, wherein
R₃ₐ, and R_{3b} are independently selected from the group consisting of:
(a) hydrogen,
(b) substituted or unsubstituted alkyl,
(c) substituted and unsubstituted aryl,
(d) substituted and unsubstituted heteroaryl,
(e) substituted and unsubstituted heterocyclyl, and
(f) substituted and unsubstituted cycloalkyl; and
R₄ is selected from the group consisting of
(1) hydrogen, and
(2) halogen,
or a pharmaceutically acceptable salt thereof, and (b) a 17α-Hydroxylase/C_{17,20}-lyase inhibitor or pharmaceutically acceptable salt thereof.

2. A combination according to claim 1, wherein the phosphatidylinositol 3-kinase inhibitor is 2-methyl-2-[4-(3-methyl-2-oxo-8-quinolin-3-yl-2,3-dihydro-imidazo[4,5-c]quinolin-1-yl)-phenyl]-propionitrile (COMPOUND A), 8-(6-methoxy-pyridin-3-yl)-3-methyl-1-(4-piperazin-1-yl-3-trifluoromethyl-phenyl)-1,3-dihydro-imidazo[4,5-c]quinolin-2-one (COMPOUND B) or a pharmaceutically acceptable salt thereof.

3. A combination according to claim 1, wherein the phosphatidylinositol 3-kinase inhibitor is 5-(2,6-di-morpholin-4-yl-pyrimidin-4-yl)-4-trifluoromethyl-pyridin-2-ylamine (COMPOUND C) or a pharmaceutically acceptable salt thereof.

4. A combination according to claim 1 wherein the 17α-Hydroxylase/C_{17,20}-lyase inhibitor is selected from the group consisting of ketoconazole, abiraterone acetate, abiraterone, 1-(2-Chloro-pyridin-4-yl)-3-(4-methyl-pyridin-3-yl)-imidazolidin-2-one or a pharmaceutically acceptable salt thereof.

5-6. (canceled)

7. A combination according to claim 1 further comprising at least one additional therapeutic agent.

8. A combination according to claim 7 wherein the at least one additional therapeutic agent is a steroid selected from the group consisting of hydrocortisone, dexamethasone, prednisolone, prednisone and combinations thereof, and any pharmaceutically acceptable salt thereof.

9. A pharmaceutical composition comprising a combination according to any one of claims 1 to 4 and at least one pharmaceutically acceptable carrier.

10. A method of treating a patient having a tumor disease which comprises administering to a subject in need thereof a combination comprising a phosphatidylinositol 3-kinase inhibitor selected from the group consisting of 2-methyl-2-[4-(3-methyl-2-oxo-8-quinolin-3-yl-2,3-dihydro-imidazo[4,5-c]quinolin-1-yl)-phenyl]-propionitrile (COMPOUND A), 8-(6-methoxy-pyridin-3-yl)-3-methyl-1-(4-piperazin-1-yl-3-trifluoromethyl-phenyl)-1,3-dihydro-imidazo[4,5-c]quinolin-2-one (COMPOUND B), and 5-(2,6-di-morpholin-4-yl-pyrimidin-4-vl)-4-trifluoromethyl-pyridin-2-ylamine (COMPOUND C) or a pharmaceutically acceptable salt thereof and (b) a 17α-Hydroxylase/C_{17,20}-lyase inhibitor or pharmaceutically acceptable salt thereof in a quantity which is jointly therapeutically effective against said tumor disease.

11. (canceled)

12. A method according to claim 10, wherein the tumor disease is prostate.

13. (canceled)

14. A method according to claim 10, wherein the amount of (a) and the amount of (b) are in a single formulation or unit dosage form.

15. A method according to claim 10, wherein the amount of (a) and the amount of (b) are in a separate formulations or unit dosage forms.

16. A method according to claim 10 which further comprises administering to a patient in need thereof a combination according to claim 10 in combination with at least one additional therapeutic agent.

17. (canceled)

18. A method according to claim 16 wherein the additional therapeutic agent is a steroid selected from the group consisting of hydrocortisone, dexamethasone, prednisolone, prednisone and combinations thereof, and any pharmaceutically acceptable salt thereof.

19. (canceled)

20. A method according to claim 10, wherein the tumor disease is selected from the group consisting of benign or malignant tumors, carcinoma of the brain, kidney, liver, bladder, breast, gastric, ovaries, colon, rectum, prostate, pancreas, lung, vagina or thyroid, or gastrointestinal cancers.
